(19) United States
(12) Patent Application Publication
(10) Pub. No.: US 2025/0228605 A1
(43) Pub. Date: Jul. 17, 2025

(54) INSTRUMENT END EFFECTOR WITH MULTIFUNCTION MEMBER AND RELATED DEVICES, SYSTEMS AND METHODS

(71) Applicant: INTUITIVE SURGICAL OPERATIONS, INC., Sunnyvale, CA (US)

(72) Inventor: Matthew Aaron Wixey, Rochester, CT (US)

(73) Assignee: INTUITIVE SURGICAL OPERATIONS, INC., Sunnyvale, CA (US)

(21) Appl. No.: 18/691,140

(22) PCT Filed: Sep. 9, 2022
(86) PCT No.: PCT/US2022/043046

§ 371 Date: Mar. 12, 2024

Related U.S. Application Data
(60) Provisional application No. 63/243,249, filed on Sep. 13, 2021.

(51) Int. Cl. A61B 18/14; A61B 17/29; A61B 18/00; A61B 18/12

(52) U.S. Cl. CPC A61B 18/1445; A61B 2017/2929; A61B 2017/2943; A61B 2018/00428; A61B 2018/00601; A61B 2018/0063; A61B 2018/126; A61B 2018/1455; A61B 2018/1467

(57) ABSTRACT

An instrument may comprise a shaft and an end effector comprising a jaw mechanism coupled to the shaft. The jaw mechanism may comprise a first jaw member and a second jaw member movable relative to one another by pivoting about a pivot axis between an open configuration of the jaw mechanism and a closed configuration of the jaw mechanism. The second jaw member may be rotatable about a roll axis of the second jaw member between at least two orientations, the roll axis of the second jaw member being transverse to the pivot axis. In a first orientation of the at least two orientations, the jaw mechanism is configured to perform a first function. In a second orientation of the at least two orientations, the jaw mechanism is configured to perform a second function differing from the first function.

### CROSS-REFERENCE TO RELATED APPLICATION

This application claims priority to U.S. Provisional Application No. 63/243,249 (filed Sep. 13, 2021), titled “INSTRUMENT END EFFECTOR WITH MULTIFUNCTION MEMBER AND RELATED DEVICES, SYSTEMS AND METHODS,” the entire contents of which are incorporated by reference herein.

### FIELD

Aspects of this disclosure relate generally to instrument end effectors and related devices, systems, and methods, for example, for use in computer-assisted teleoperated manipulator systems. More specifically, aspects of the disclosure relate to end effectors capable of performing multiple functions, and remotely-controlled instruments including such end effectors.

### INTRODUCTION

Remotely-controlled instruments generally comprise end effectors, which are often disposed at a distal end portion of the instrument and comprise one or more functional elements, such as, for example, a jaw mechanism, a stapler, a blade, a camera, an electrode, a sensor, etc., to perform one or more functions of the instrument, such as cutting, sealing, grasping, imaging, etc. The functions performed by an end effector may be controlled and driven by the forces, torques, other drive inputs, and/or flow function inputs (e.g., electrical energy, illumination, irrigation, etc.) received by the instrument via various interfaces generally located at a proximal end portion of the instrument. In some such instruments, actuation elements run from the proximal end portion along an instrument shaft to transmit forces and/or other functionality from a transmission mechanism at the proximal end portion of the instrument to the end effector. Such remotely-controlled instruments can be manually operated, for example, via a manually-actuated inputs at a handle or other interface mounted at the proximal end portion. Alternatively, such remotely-controlled instruments may be coupled to or configured to be coupled to computer-assisted manipulator systems, which may be operably coupled to a remotely located console that provides the interface to receive input from a user.

Computer-assisted manipulator systems (“manipulator systems”), sometimes referred to as robotically assisted systems or robotic systems, may comprise one or more manipulators that can be operated with the assistance of an electronic controller (e.g., computer) to move and control functions of one or more instruments when coupled to the manipulators. A manipulator generally comprises a plurality of mechanical links connected by joints. An instrument is removably couplable to (or permanently coupled to) one of the links, typically a distal link of the plural links. The joints are operable to cause the links to move (i.e., rotate and/or translate) relative to one another, imparting various degrees of freedom to the manipulator to enable the manipulator to move the instrument around a worksite. The manipulators of a manipulator system can be used to transmit a variety of forces and torques to the instruments to perform various procedures, such as medical procedures or non-medical procedures (e.g., industrial procedures). The link to which the instrument is couplable or coupled (e.g., an instrument carriage) comprises drive outputs to interface with and mechanically transfer driving forces to corresponding drive inputs of the instrument to control degrees of freedom of motion and/or other functions of the instrument. Electrical power, data signals, vacuum suction, insufflation, irrigation, and/or other useful flows may also be transferred to the instrument via various interfaces, which may include interfaces of the manipulator or interfaces of other parts or subsystems to which the instrument may be operably couplable or coupled (e.g., an auxiliary system). As mentioned above, the manipulator system may be operably coupled (e.g., through a controller) to a console with user input devices which register user inputs and control operations of the system based on the inputs. In some cases, an input device may be arranged such that as the input device is actuated, the instrument is controlled to follow or mimic the movement of the input device, which may provide the user a sense of directly controlling the instrument.

One type of remotely-controlled instruments is a medical instrument, which may be used to perform medical procedures, such as, for example, surgical, diagnostic, or therapeutic procedures. Medical instruments may include a variety of instruments used to perform medical procedures, such as therapeutic instruments, diagnostic instruments, surgical instruments, and/or imaging instruments. In some examples, the medical instruments may be inserted into a patient through a natural orifice or an incision (including through a port or other guide inserted in the incision). Such instruments that are remotely controlled may be particularly useful, for example, in performing minimally invasive surgical procedures. A minimally invasive surgical procedure may be designed to reduce the amount of tissue that is damaged during a surgical procedure, for example by decreasing the number and/or size of incisions through which medical instruments are inserted.

In some circumstances during a medical procedure, different functions may be performed. For example, in a medical procedure, it may be desirable to both cut and seal tissue, or to apply different types of electrosurgical energy. Still other procedures may utilize different sensed conditions and then perform a function based on that sensed condition. In such cases, if different instruments are used to perform the different functions, a relatively more invasive procedure may result, such as the insertion of two instruments at the same time and/or the consecutive insertion and withdrawal of different instruments. Accordingly, some procedures may benefit from an instrument capable of performing multiple functions. However, challenges can arise in providing instruments having end effectors capable of performing multiple functions (multi-function end effector), including the space that may be needed to provide multiple functions and/or to actuate those functions. Also, such multi-function instruments are relatively more complex in their structure and systems needed to actuate the various functionality, which can result in increased cost and decreased life expectancy of the instrument. Further, it can be difficult to provide an instrument to perform the various respective functions in a reliable and robust manner.

Accordingly, a need exists to provide instruments with multifunction end effectors that are capable of performing multiple functions with high effectiveness while still being relatively simple and compact, and/or to otherwise improve performance of instrument end effectors.

### SUMMARY

Various embodiments of the present disclosure may solve one or more of the above-mentioned problems and/or may demonstrate one or more of the above-mentioned desirable features. Other features and/or advantages may become apparent from the description that follows.

In accordance with at least one embodiment of the present disclosure an instrument may comprise a shaft and an end effector comprising a jaw mechanism coupled to the shaft. The jaw mechanism may comprise a first jaw member and a second jaw member movable relative to one another by pivoting about a pivot axis between an open configuration of the jaw mechanism and a closed configuration of the jaw mechanism. The second jaw member may be rotatable about a roll axis of the second jaw member between at least two orientations, the roll axis of the second jaw member being transverse to the pivot axis. In a first orientation of the at least two orientations, the jaw mechanism is configured to perform a first function. In a second orientation of the at least two orientations, the jaw mechanism is configured to perform a second function differing from the first function.

In accordance with another embodiment of the present disclosure an instrument may comprise a shaft and a jaw mechanism coupled to the shaft. The jaw mechanism may comprise a first jaw member and a second jaw member movable relative to one another by pivoting about a pivot axis between an open configuration of the jaw mechanism and a closed configuration of the jaw mechanism. The second jaw member is rotatable relative to the shaft about a roll axis of the second jaw member and independently of the first jaw member, the roll axis of the second jaw member being transverse to the pivot axis.

In accordance with another embodiment of the present disclosure an instrument may comprise a shaft and an end effector coupled to the shaft. The end effector may comprise a multifunction member comprising a proximal end, a distal end, an outer peripheral surface, and a longitudinal axis extending between the proximal end and the distal end. The multifunction member is rotatable relative to the shaft about the longitudinal axis. The multifunction member comprises a first functional feature configured to perform a first function and a second functional feature configured to perform a second function. The first functional feature and the second functional feature are positioned on the outer peripheral surface at different angular positions around the longitudinal axis.

In accordance with another embodiment of the present disclosure an instrument may comprise a shaft, an actuatable element extending through the shaft, and an instrument transmission system coupled to the shaft. The instrument transmission system may comprise a chassis, a first drive input, a second drive input, a first gear assembly operably coupled to and driven by the first drive input, and a second gear assembly operably coupled to and driven by the second drive input. Rotation of the first gear assembly and the second gear assembly relative to the chassis at a same angular velocity causes rotation of the actuatable element relative to the shaft. Rotation of the first gear assembly relative to the chassis while the second gear assembly is held stationary relative to the chassis causes translation of the actuatable element relative to the shaft.

In accordance with another embodiment of the present disclosure a method of using an instrument that has a jaw mechanism may comprise, while a first jaw member of the jaw mechanism is in a first orientation, causing the first jaw member to perform a first function on material grasped by the jaw mechanism. The method may further comprise rotating the first jaw member from the first orientation to a second orientation about a rotation axis extending from a proximal end of the first jaw member to a distal end of the first jaw member. The method may further comprise, while the first jaw member is in the second orientation, causing the first jaw member to perform a second function, different from the first function, on material grasped by the jaw mechanism.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1 is a schematic view of an embodiment of a computer-assisted medical system.

FIG. 2 is a schematic view of an embodiment of an instrument.

FIG. 3 is a perspective view of an embodiment of an end effector of an instrument.

FIG. 4A is a perspective view from a distal end of the end effector of FIG. 3 in a first configuration.

FIG. 4B is a perspective end view from a distal end of the end effector of FIG. 3 in a second configuration.

FIG. 5 is a perspective view of the end effector of FIG. 3 showing interior portions thereof.

FIG. 6 is a perspective section view of the end effector of FIG. 3 with the section taken along A in FIG. 4A.

FIG. 7 is a perspective view of a coupling mechanism of the end effector of FIG. 3.

FIG. 8 is a perspective section view of a rotational coupling of the coupling mechanism of FIG. 7, with the section taken along B in FIG. 7.

FIG. 9 is a cross-sectional view of an embodiment of a multifunction jaw member, with the section being transverse to a longitudinal axis of the multifunction jaw member.

FIG. 10 is a cross-sectional view of another embodiment of a multifunction jaw member, with the section being transverse to a longitudinal axis of the multifunction jaw member.

FIG. 11 is a cross-sectional view of yet another embodiment of a multifunction jaw member, with the section being transverse to a longitudinal axis of the multifunction jaw member.

FIG. 12 is perspective view of an embodiment of an instrument force transmission system.

FIG. 13 is perspective view of the instrument force transmission system of FIG. 12 with an outer housing thereof removed to show internal components.

FIG. 14 is perspective view of the instrument force transmission system of FIG. 13 with the section taken along B in FIG. 13.

FIG. 15 is a perspective view of another embodiment of an end effector of an instrument.

FIG. 16 is a perspective view of yet another embodiment of an end effector of an instrument

### DETAILED DESCRIPTION

As noted above, it may be desirable under some circumstances for an end effector of an instrument to be capable of performing multiple functions. With multiple functions being performed by one end effector, it may be possible to avoid (or reduce the frequency of) switching between different instruments during a procedure, thus reducing the complexity and duration of the procedure. In addition, using a multifunction end effector may reduce the number of instruments that are needed for a given procedure, as functions previously performed by multiple instruments are now performed by one. This reduction in the number of instruments reduces costs, as fewer instruments need be purchased, and may also reduce the amount of space that is occupied in the worksite during the procedure, as the space previously occupied by multiple instruments may now be occupied by one. In addition, this reduction in the amount of space occupied may allow for a reduction in size of the worksite and/or in the number of access points to a worksite (e.g., use of smaller or fewer number of incisions in medical procedures).

However, as also noted above, existing instruments with multifunction end effectors can be relatively large, relatively complex, and/or relatively less effective at performing one or more of their functions. As a non-limiting example, in the context of medical procedures, there exist various multifunction end effectors for transecting vessels (e.g., blood vessels). Such end effectors for transecting vessels may provide both a vessel cutting function and a vessel sealing function. However, these vessel cutting and sealing end effectors may be relatively larger than is desired in some circumstances, relatively more complex than is desired in some circumstances, and/or not as robust at sealing and/or cutting as is desired in some circumstances.

For example, one type of end effector for transecting vessels has a jaw mechanism comprising two electrodes for performing a bipolar electrical sealing function and a movable (e.g., translating) blade for performing a mechanical cutting function. Such an end effector seals a vessel by grasping the vessel between opposing jaws that have an electrode grasping surface (e.g., the jaw itself may be an electrode or may support an electrode) and passing electrical energy between the electrodes and through the tissue to seal (e.g., coagulate) a portion of the vessel. The end effector also can perform a cutting function, for example after sealing, by passing the movable blade through the sealed portion of the vessel still grasped between the jaws. The movable blade and the mechanism that drives the blade can result in the end effector being relatively large and mechanically complex. Moreover, the blade may dull over time and the end effector may otherwise experience wear due to the sliding function of the blade, which can reduce the lifespan for the end effector.

Another type of end effector for transecting vessels uses bipolar electrical energy passed between the opposing electrodes of jaw members for both the sealing and cutting functions. The end effector may seal and cut a vessel grasped between the electrodes by alternately applying different modes of electrical energy between the electrodes at controlled timings so as to cut and seal the vessel grasped between the jaws supporting the electrodes. Such end effectors may be relatively small and mechanically less complex (e.g., fewer moving parts), as compared to the end effectors comprising the moving blade to perform the cutting function. However, to obtain simultaneous sealing and cutting, a relatively complex control system may be needed to precisely control the modes of energy and the timings of their application. Moreover, the speed and/or effectiveness of the sealing and/or cutting functions can pose challenges because the sealing and cutting operations may have different optimal electrode configurations. But because the electrodes in these end effectors are used for both sealing and cutting, they may not be optimally configured to perform either or both functions.

To address some (or all) of the issues noted above and otherwise improve instrument end effectors, in embodiments disclosed herein an end effector is provided that is capable of performing multiple functions while also being relatively compact, relatively simple, and relatively effective in performing the functions. Moreover, in embodiments disclosed herein, end effectors may be actuatable through robust drive mechanisms that offer ease in use and manufacture, and that provide durability and reliability, while maintaining relatively low space requirements.

According to various embodiments disclosed herein, an end effector comprises a jaw mechanism with two opposing jaw members. One of the jaw members (referred to herein as a “multifunction jaw member”) has multiple different functional features located at different positions on the jaw member, with each functional feature being a part of the multifunction jaw member that is configured to facilitate the performance of a particular function of the instrument. For example, functional features could include but are not limited to: an electrode having a first shape (e.g., a shape adapted to perform bipolar sealing), an electrode having a second shape (e.g., a shape adapted to perform bipolar cutting), a surface adapted for grasping (e.g., a surface with friction enhancing features), a blade for mechanical cutting, etc. In embodiments disclosed herein, the multifunction jaw member is capable of being reconfigured during usage to change which one of the functional features is positioned for active use. In some embodiments, the functional feature that is positioned for active use can be changed by changing the pose of the multifunction jaw member, which may include, for example rotating the multifunction jaw member. For example, a given functional feature may be usable to perform its respective function when it is located in an “active position,” such as a position in which the functional feature faces the other jaw member of the jaw mechanism. The multifunction jaw member may be movable between various poses in which different functional features are in the active position.

In some embodiments, the functional features are arranged around a peripheral surface of the jaw member, such as at different angular positions relative to a longitudinal axis of the jaw member. In some of these embodiments, the multifunction jaw member, which extends distally from a shaft of the instrument, is rotated about a longitudinal axis of the jaw member to bring different functional features at different positions around the peripheral surface into the active position. In some embodiments, the longitudinal axis of the multifunction jaw member extends generally parallel to a longitudinal axis of the instrument shaft. Thus, by rotating the multifunction jaw member between different orientations, the end effector can be selectively reconfigured, including during a medical procedure, between multiple different functions so as to permit more than one function to be performed without the need to exchange instruments.

In some embodiments, one of the functional features of the multifunction jaw member comprises a sealing electrode configured for use in bipolar electrical sealing, and another of the functional features comprises a cutting electrode configured for use in bipolar electrical cutting. In such embodiments, the end effector can be configured for a bipolar electrical sealing function by moving the multifunction jaw member into a first pose (e.g., rotating the multifunction jaw member about a longitudinal axis of the jaw member to a first orientation) in which the sealing electrode is in the active position, and the end effector can be reconfigured for a bipolar electrical cutting function by moving the multifunction jaw member to a second pose (e.g., rotating the multifunction jaw member to a second orientation) in which the cutting electrode is in the active position. In some embodiments, the active positions of the sealing and cutting electrodes of the multi-function jaw member correspond to position in which the respective sealing or cutting electrode is opposite from another electrode of the opposing jaw member.

In embodiments disclosed herein, the end effector is relatively compact. In particular, because multiple functional features are provided as parts of the same jaw member, at least some of their structure is shared in common, thus reducing the amount of space that is needed as compared to a configuration in which each functional feature were provided as a separate component of the end effector.

Moreover, the end effectors of embodiments disclosed herein may be relatively effective at performing their intended functions. In some applications, the effectiveness with which a functional feature performs a given function may depend on the configuration (e.g., shape and/or size) of the functional feature, with some configurations being more effective than others. For example, an electrode having a relatively broad contact surface may be more effective at performing a bipolar sealing function than an electrode with a relatively narrow contact surface, while the opposite may be true for a bipolar cutting function. In embodiments disclosed herein, each functional feature can be specialized for a particular function, and therefore each functional feature may be provided with a configuration that promotes its effectiveness at performing its intended function. For example, a functional feature for bipolar sealing may comprise an electrode with a relatively broad tissue contact surface, while a functional feature for bipolar cutting may comprise an electrode with a relatively narrow tissue contact surface. The ability of disclosed embodiments to provide different electrode configurations that are better suited for performing different respective functions thus alleviates the need to choose a single electrode configuration to perform both functions, in which case one function may be optimized to a detriment of the other or neither function is optimized and thus neither is performed as well.

Furthermore, in embodiments disclosed herein, the end effectors may be relatively simple in terms of construction and use. The end effectors can have relatively few moving parts and relatively uncomplicated control schemes. This can result in the end effectors being more robust, more precise, and/or having smaller overall dimensions.

In the description above and with regard to various figures below, cutting and sealing electrodes are described as two non-limiting examples of the functional features of the multi-function end effector member. But those having ordinary skill in the art would appreciate that the present disclosure is not limited to those particular functional features and that other types of functional features may be included in the multi-function jaw member, in addition to or in lieu of the sealing and cutting electrodes. Moreover, any number of two or more functional features may be included. Examples of other functional features, which are described in greater detail below, include mechanical cutting features (e.g., a mechanical blade), grasping features (e.g., a surface with friction enhancing features), a light emitting device, an optical device (e.g., a lens), etc.

Turning now to the figures, various embodiments will be described in greater detail.

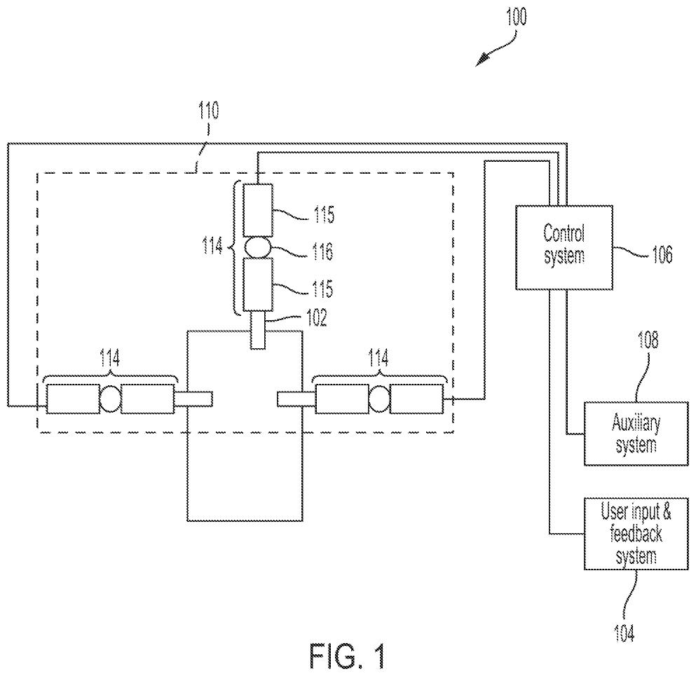

FIG. 1 is a schematic block diagram of an embodiment of a computer-assisted system 100 for remote control of instruments. The system 100 comprises a manipulator assembly 110, a control system 106, and a user input and feedback system 104. The system 100 may also include an auxiliary system 108. These components of the system 100 are described in greater detail blow.

The manipulator assembly 110 comprises one or more manipulators 114. FIG. 1 illustrates three manipulators 114, but any number of manipulators 114 may be included. While a manipulator may comprise a single mechanical link, in the embodiment of FIG. 1, each manipulator 114 comprises a kinematic structure of two or more links 115 coupled together by one or more joints 116. The joints 116 may impart various degrees of freedom of movement to the manipulator 114, allowing the manipulator 114 to be moved around a workspace. For example, some joints 116 may provide for rotation of links 115 relative to one another, other joints 116 may provide for translation of links 115 relative to one another, and some may provide for both rotation and translation. Some or all of the joints 116 may be powered joints, meaning a powered drive element may control movement of the joint 116 through the supply of motive power. Such drive elements may comprise, for example, electric motors, pneumatic or hydraulic actuators, etc. Additional joints 116 may be unpowered joints. In addition to drive elements that control the joints 116, the manipulator 114 may also include drive elements (not illustrated) that drive inputs of the instrument 102 to control operations of the instrument, such as moving an end-effector of the instrument, opening/closing jaws, driving translating and/or rotating components, etc. In some embodiments, the manipulator assembly can include flux delivery transmission capability as well, such as, for example, to supply electricity, fluid, vacuum pressure, light, electromagnetic radiation, etc. to the end effector. In other embodiments, such flux delivery transmission may be provided to an instrument through another auxiliary system, described further below. FIG. 1 illustrates each manipulator 114 as having two links 115 and one joint 116, but in practice a manipulator may include more links 115 and more joints 116, depending on the needs of the system 100. The more links 115 and joints 116 are included, the greater the degrees of freedom of movement of the manipulator 114.

Each manipulator 114 may be configured to support and/or operate one or more instruments 102. In some examples the instruments 102 may be fixedly coupled to the manipulator 114, while in other examples one of the links 115 may be configured to have one or more separate instruments 102 removably coupled thereto. The instruments 102 may include any tool or instrument, including for example industrial instruments and medical instruments (e.g., surgical instruments, imaging instruments, diagnostic instruments, therapeutic instruments, etc.).

The system 100 can also include a user input and feedback system 104 operably coupled to the control system 106. The user input and feedback system 104 comprises one or more input devices to receive input control commands to control operations of the manipulator assembly 110. Such input devices may include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device may be provided with the same degrees of freedom as the associated instrument that they control, and as the input device is actuated, the instrument, through drive inputs from the manipulator assembly, is controlled to follow or mimic the movement of the input device, which may provide the user a sense of directly controlling the instrument. Telepresence input devices may provide the operator with telepresence, meaning the perception that the input devices are integral with the instrument. The user input and feedback system 104 may also include feedback devices, such as a display device (not shown) to display images (e.g., images of the worksite as captured by one of the instruments 102), haptic feedback devices, audio feedback devices, other graphical user interface forms of feedback, etc.

The control system 106 may control operations of the system 100. In particular, the control system 106 may send control signals (e.g., electrical signals) to the manipulator assembly 110 to control movement of the joints 116 and to control operations of the instruments 102 (e.g., through drive interfaces at the manipulators 114). In some embodiments, the control system 106 may also control some or all operations of the user input and feedback system 104, the auxiliary system 108, or other parts of the system 100. The control system 106 may include an electronic controller to control and/or assist a user in controlling operations of the manipulator assembly 110. The electronic controller comprises processing circuitry configured with logic for performing the various operations. The logic of the processing circuitry may comprise dedicated hardware to perform various operations, software (machine readable and/or processor executable instructions) to perform various operations, or any combination thereof. In examples in which the logic comprises software, the processing circuitry may include a processor to execute the software instructions and a memory device that stores the software. The processor may comprise one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In examples in which the processing circuitry includes dedicated hardware, in addition to or in lieu of the processor, the dedicated hardware may include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry may also include any combination of dedicated hardware and processor plus software.

As noted above, differing degrees of user control versus autonomous control may be utilized in the system 100, and embodiments disclosed herein may encompass fully user-controlled systems, fully autonomously-controlled systems, and systems having any combination of user and autonomous control. For operations that are user-controlled, the control system 106 generates control signals in response to receiving a corresponding user input command via the user input and feedback system 104. For operations that are autonomously controlled, the control system 106 may execute pre-programmed logic (e.g., a software program) and may determine and send control commands based on the programming (e.g., in response to a detected state or stimulus specified in the programming). In some systems, some operations may be user controlled and others autonomously controlled. Moreover, some operations may be partially user controlled and partially autonomously controlled-for example, a user input command may initiate performance of a sequence of events, and then the control system 106 may perform various operations associated with that sequence without needing further user input.

The auxiliary system 108 may comprise various auxiliary devices that may be used in operation of the system 100. For example, the auxiliary system 108 may include power supply units, auxiliary function units (e.g., functions such as irrigation, evacuation, energy supply, illumination, sensors, imaging, etc.). As one example, in a system 100 for use in a medical procedure context, the auxiliary system 108 may comprise a display device for use by medical staff assisting a procedure, while the user operating the input devices may utilize a separate display device that is part of the user input and feedback system 104. As another example, in a system 100 for use in a medical context, the auxiliary system 108 may comprise flux supply units that provide surgical flux (e.g., electrical power) to instruments 102. An auxiliary system 108 as used herein may thus encompass a variety of components and does not need to be provided as an integral unit.

As noted above, one or more instruments 102 can be mounted to the manipulator 114. In some embodiments, an instrument carriage physically supports the mounted instrument 102 and has one or more actuators (not illustrated) to provide driving forces to the instrument 102 to control operations of the instrument 102. The actuators may provide the driving forces by actuating drive outputs (not illustrated), such as rotary disc outputs, joggle outputs, linear motion outputs, etc. The drive outputs may interface with and mechanically transfer driving forces to corresponding drive inputs of the instrument 102 (directly, or via intermediate drive outputs, which may be part of a sterile instrument adaptor (ISA) (not illustrated)). The ISA may be placed between the instrument 102 and the instrument carriage to maintain sterile separation between the instrument 102 and the manipulator 114. The instrument carriage may also comprise other interfaces (not illustrated), such as electrical interfaces to provide and/or receive electrical signals to/from the instrument 102.

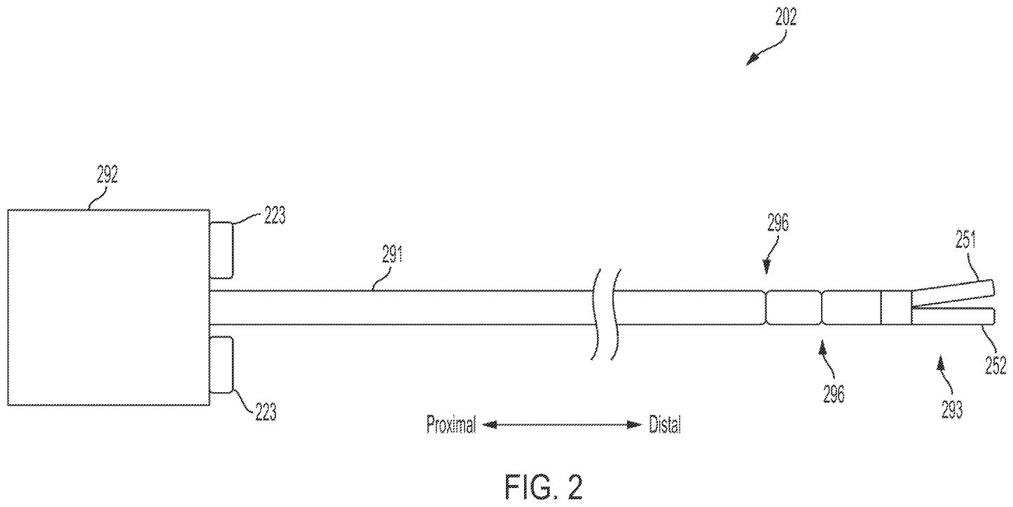

FIG. 2 is a schematic diagram illustrating an embodiment of an instrument 202. In some embodiments, the instrument 202 may be used and controlled via a computer-controlled system, such as the system 100. For example, the instrument 202 may be used as the instrument 102. In other embodiments, the instrument 202 may a manually operable instrument.

As shown in FIG. 2, the instrument 202 comprises a shaft 291 and an end effector 293 attached to the shaft 291 at a distal end portion of the instrument 202, with proximal and distal directions referenced herein illustrated in FIG. 2. The end effector 293 is configured to perform two or more functions, as described in greater detail below. The instrument 202 may also comprise a force transmission system 292, which in some embodiments, may be located at a proximal end portion of the instrument 202. The force transmission system 292 is attached to the shaft 291 and comprises one or more drive inputs 223 configured to receive driving forces and/or other inputs that control functions of the instrument 202, such as movements of the instrument 202 and/or functions of the end effector 293.

The end effector 293 of the instrument 202 is capable of performing at least two functions, as described above. In some embodiments the end effector 293 comprises a multifunction member 252 having at least two functional features to perform different functions. As described above, a functional feature is a part of the multifunction member 252 that is configured to facilitate the performance of a particular function of the instrument. For example, a functional feature may be a part of the member 252 that has a distinguishing characteristic (e.g., particular shape, size, sharpness, and/or other functional capability (e.g., electrical conductivity) that makes the feature useful for performing a particular function. For example, a functional feature for a bipolar sealing function may comprises an electrode (a piece of conductive material coupled to a power source) shaped to have a relatively broad contact surface (surface exposed to come into contact with tissue), while a functional feature for a bipolar cutting function may comprise an electrode having a relatively narrow contact surface. As another example, a functional feature for mechanical cutting may comprise a sharp edge. As another example, a functional feature for securely grasping objects may comprise a contact surface with friction enhancing features (e.g., grooves, protrusions, roughening elements, knurling, etc.). Other examples of functional features include a monopolar electrode (e.g., for monopolar sealing and/or monopolar cutting), a stapler for firing staples, an anvil for closing staples, a lens of a camera system, and an inlet/outlet of a flow removal/delivery system such as a vacuum suction system or fluid (gas or irrigation) delivery system. The functional features may be integral with a main body of the multifunction member 252, or the functional features may be distinct from and coupled to the main body of the multifunction member 252. For example, in some embodiments in which the functional features comprise one or more electrodes, the electrodes may be formed from different portions of the main body of the multifunctional member 252, such as different faces or surface features of the multifunctional member 252, which are all integral with one another (in which case a main body of the multifunctional member 252 may be a conductive material). Thus, in such embodiments the electrodes of the multifunction member 252 may all be electrically coupled together. In other embodiments, the electrodes may be separate parts that are coupled to the multifunctional member 252. Similarly, an electrode of an opposing member (if present) may be integral with a main body of the opposing member, or may be a separate part that is coupled to the main body.

In some embodiments, the end effector 293 comprises a functional member 251 in addition to the multifunction member 252. In some of embodiments, the multifunction member 252 is movable relative to the rest of the end effector 293 (i.e., relative to the other member 251), such that the pose (position and/or orientation) of the multifunction member 252 can be changed. The pose of the multifunction member 252 can be changed such that each of the functional features of the multifunction member 252 is brought selectively into an active position. The active position is a position in which the functional feature is ready to perform the functional feature's corresponding function. For example, the active position may be a position in which the functional feature is located opposite from and facing the other member 251. Thus, by moving different functional features into the active position, the end effector 293 can be reconfigured to perform different functions.

In some embodiments, the end effector 293 comprises a jaw mechanism and the members 251 and 252 are jaw members of the jaw mechanism that can open and close relative to each other, with one or both jaw members configured to move to open and close the jaw mechanism. In such embodiments, the active position may be a position in which the functional feature is located opposite from and facing the other member 251 such that the functional feature can come into contact with an object that is grasped by the jaw mechanism between the members 251 and 252.

In some embodiments, the changing of the pose of the multifunction member 252 comprises rotating (rolling) the multifunction member 252 about a roll axis of the member (e.g., a longitudinal axis of the multifunction member 252). In such embodiments, the functional features may be arranged around a peripheral surface of the multifunction member 252, such that each functional feature is brought into the active position as the multifunction member 252 is rotated at a respectively corresponding orientation of the multifunction member 252. In some embodiments, the roll axis of the multifunction member 252 may be parallel to a longitudinal axis of the shaft 291, or in other words extending parallel to a proximal-to-distal direction. Rotation of the multifunction member 252 about the roll axis may also be referred to herein as rotation in a roll degree of freedom of motion.

The movement of the multifunction member 252 to reconfigure the end effector 293 may be driven by forces transferred from the drive inputs 223 through the instrument shaft 291 via a force transmission system. Moreover, the movement of the multifunction member 252 to reconfigure the end effector 293 may occur while using the instrument 202 during a medical procedure, as opposed to during maintenance, repair, or reprocessing. Thus, for example, a user is able to switch between performing multiple different functions during a procedure without having to change between multiple instruments and/or without having to stop the procedure to withdraw an instrument for manual reconfiguration of the end effector.

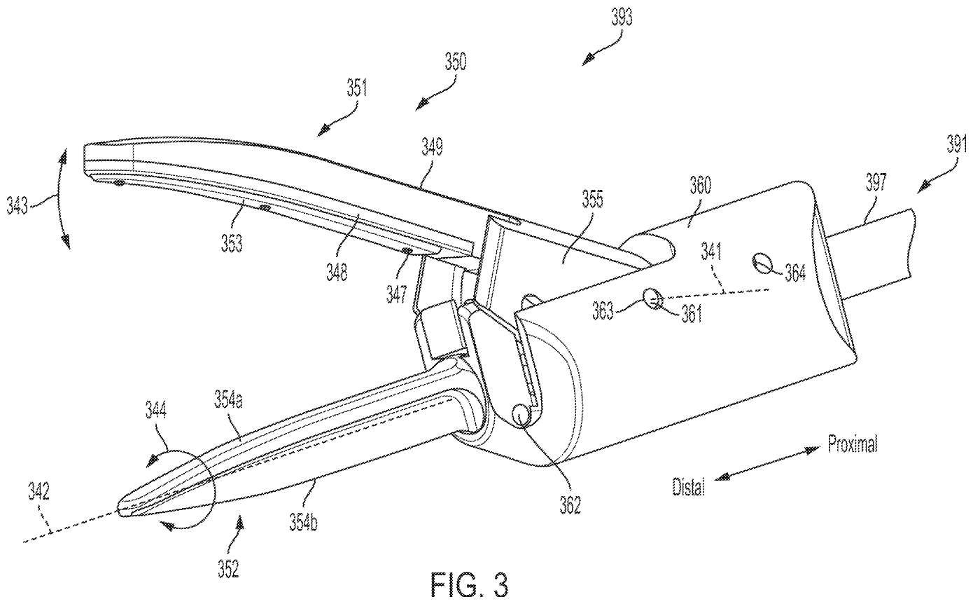

In some embodiments, the functions performed by the functional features of the multifunction member 252 comprise any combination of two or more of: bipolar electrical sealing, bipolar electrical cutting, grasping, mechanical cutting, monopolar electrical cutting, cauterization, or stapling. For example, in some embodiments, the multifunction member 252 has a first functional feature comprising a first electrode for performing bipolar sealing and a second functional feature comprising a second electrode for performing bipolar cutting. The first electrode and second electrode may be arranged at different angular orientations around a peripheral surface of the multifunction member 252 such that, by rotating the multifunction member 252 around a roll axis thereof, the first electrode and second electrode are alternatively brought into an active position opposite from another member 251 of the end effector 293. Further embodiments of the end effector 293 and functional features thereof are described in greater detail below with respect to FIGS. 3-11.

The instrument 202 may have various degrees of freedom of motion, which may include degrees of freedom of motion associated with performing the functions of the instrument 202 (e.g., opening and closing of the jaw mechanism), as well as degrees of freedom of motion to move the shaft 291 and/or the end effector 293 about the workspace and change the pose of the end effector 293 relative to the remainder of the instrument 202 (above and beyond the motion imparted by moving a manipulator to which the instrument 202 may be mounted). For example, the instrument 202 may comprise one or more joints 296, such as a wrist mechanism, to allow the end effector 293 to be moved relative to the remainder of the shaft 291, for example by changing the pitch, yaw, roll, or any combination thereof of the end effector 293. As another example, the shaft 291 may be rotatable so as to roll relative to the force transmission system 292 or otherwise have degrees of freedom of motion.

As noted above, the force transmission system 292 may comprise drive inputs 223 that interface with and are driven by the drive outputs of the manipulator (directly or via an intermediary such as an ISA). In addition, the force transmission system 292 may contain various force conversion components (not visible in FIG. 2) to convert the motion of the drive inputs 223 into motion that drives degrees of freedom of the instrument 202. Examples of drive inputs 223 include, but are not limited to, rotational couplers (discs), levers, linear motion inputs/outputs, etc. The forces and motion imparted to the drive inputs 223 may be converted by the force conversion mechanisms of the force transmission system 292 into movement of one or more actuation elements (e.g., cables, rods, etc.) (not shown in FIG. 2) that are operably coupled to the force conversion mechanisms and extend through the shaft 291, with the motion of the actuation elements controlling the degrees of freedom of motion of the instrument 202. In particular, the motion of the actuation elements may control functions of the instrument 202 and actuation of the end effector 293, such as opening/closing a jaw mechanism (if present) and rotating the multifunction member 252. Those having ordinary skill in the art are familiar with such remotely controlled instruments and the components thereof for receiving an input at a proximal end portion of the instrument and operating the instrument, including an end effector, at a distal end portion of the instrument.

The shaft 291 comprises an outer housing through which various components are routed to transmit force or other functionality to the end effector 293. For example, the housing of the shaft 291 may be shaped as a hollow tube having a central bore through which the components are routed and/or with bores around a periphery of the tube, such as in a thickness of the tube wall, through which components are routed. Components that are routed through the housing of the shaft 291 may include, for example, the actuation elements described above (e.g., push-pull, pull-pull, and/or rotary elements) to drive movement and/or actuate movement of the instrument 202, electrical power transmission lines, data communication lines, vacuum suction delivery lines, fluid delivery lines, electromagnetic energy delivery lines, etc. As noted above, in some embodiments, the shaft 291 comprises one or more joints 296, and articulation of the joints 296 may also be driven by corresponding actuation elements (e.g., cables, rods, etc.) that are routed through the shaft 291. The end effector 293 is coupled to and supported at a distal portion of the shaft 291, directly or via intermediate parts such as a wrist mechanism.

In some embodiments, the instrument 202 may be a medical instrument. Medical instruments may comprise surgical instruments (e.g., grasping instruments, cutting instruments, electrocautery instruments, stapling instruments, suturing instruments, etc.), imaging instruments (e.g., endoscopes), diagnostic instruments, and therapeutic instruments, which can have a variety of configurations, for example, with and without end effectors.

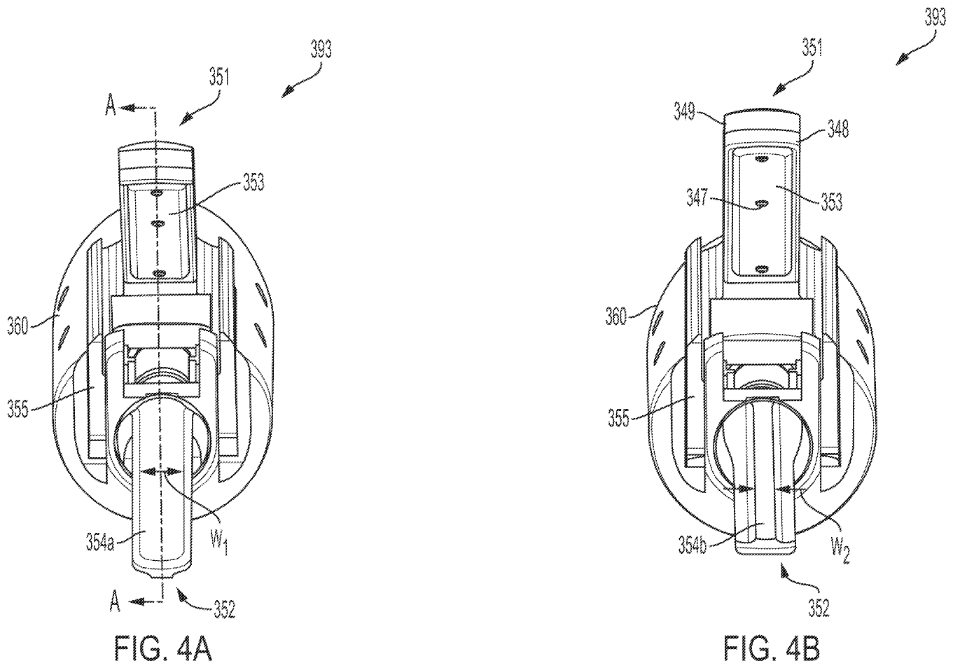

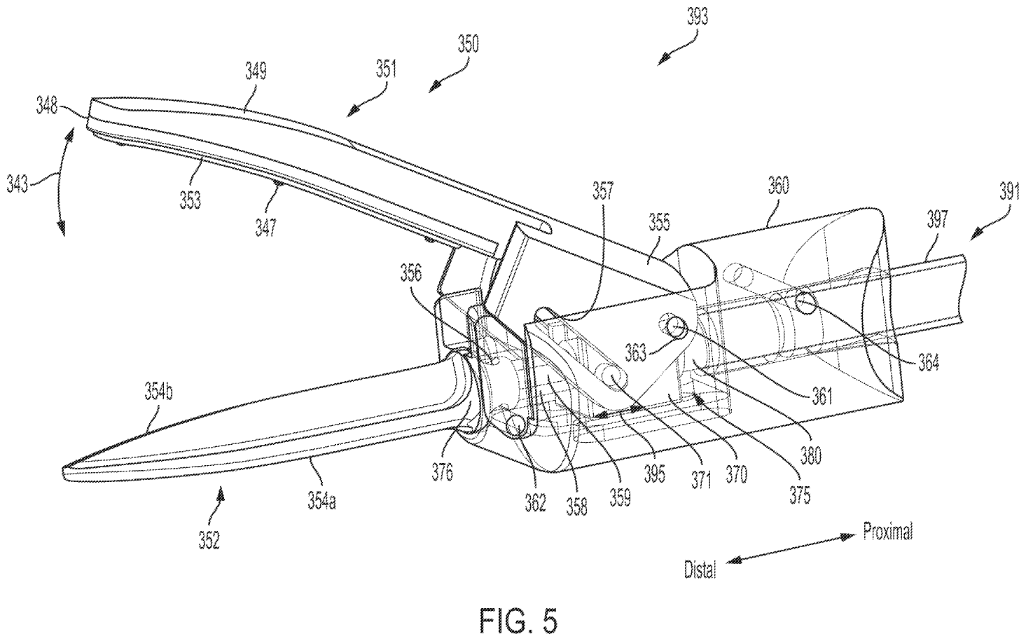

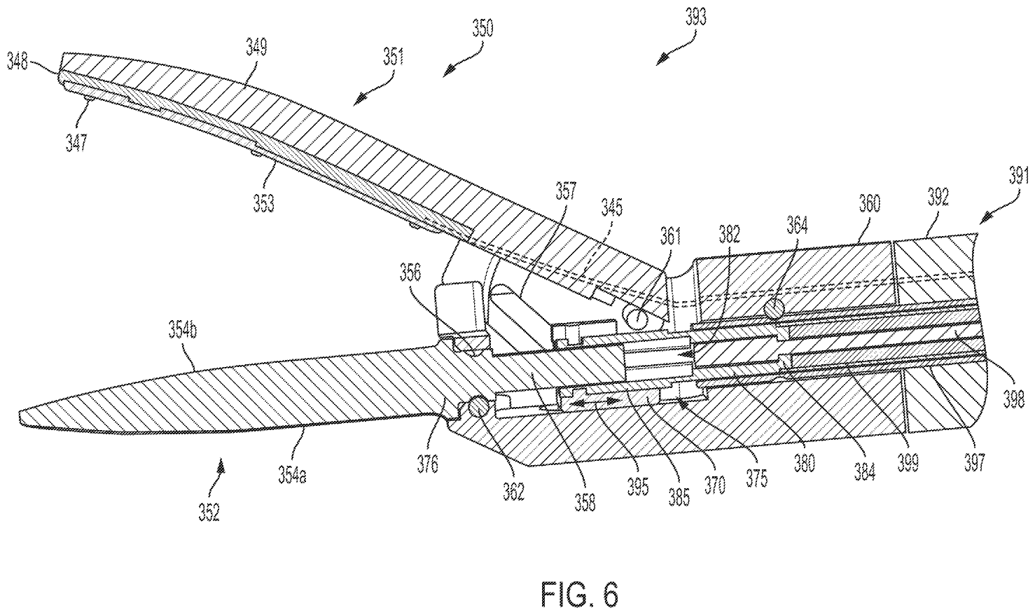

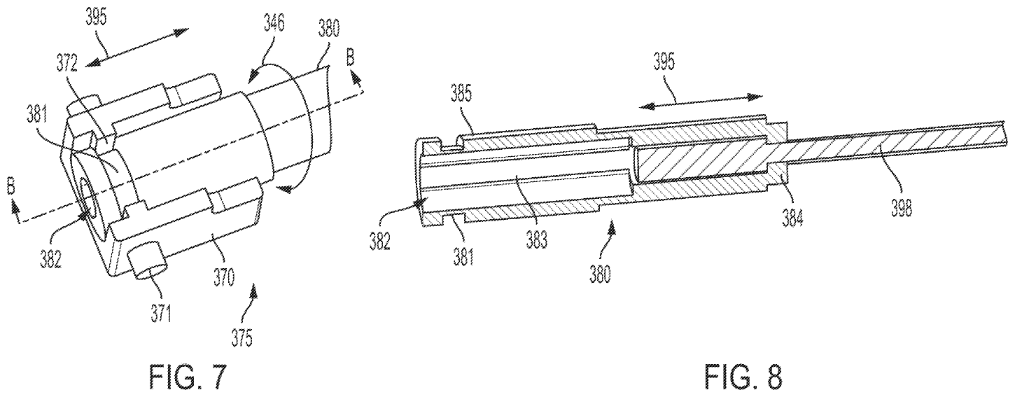

Turning now to FIGS. 3-8, an embodiment of an end effector 393 is described in greater detail. The end effector 393 may be used as the end effector 293. FIG. 3 comprises a perspective view of the end effector 393 from a point of view positioned along a lateral side of the end effector 393. FIGS. 4A and 4B are perspective views of the end effector from a point of view positioned in front of and slightly above a distal end of the end effector 393, with each illustrating different poses of a jaw member 352. FIG. 5 is a perspective view of the end effector 393 with a clevis 360 made transparent. FIG. 6 is a perspective section of the end effector 393, with the section taken along A in FIG. 3. FIG. 7 is a perspective view of a closure block 370 and rotational coupling 380 of the end effector 393. FIG. 8 is a perspective section of the rotational coupling 380, with the section taken along A in FIG. 3.

Some elements of the end effector 393 are illustrated in multiple figures. As elements of the end effector 393 are described, one or a few figures which are thought to be particularly pertinent to the aspect will be noted, but it should be understood that other figures besides those that are identified may also illustrate the same element from other perspectives. Thus, the description below will not necessarily describe the figures FIGS. 3-8 separately and in strict sequence.

As shown in FIGS. 3-6, the end effector 393 comprises a jaw mechanism 350 coupled to a clevis 360. The clevis 360 and jaw mechanism 350 are coupled to and supported at a distal portion of a shaft 391. The shaft 391 can be used as the shaft 291 described above. The shaft 391 comprises one or more housing structures (e.g., tubes, guide elements with lumens, coil pipes, etc.) that house one or more components such as actuation elements, electrical lines, airflow lines, etc. For example, as shown in FIGS. 3, 5, and 6, the shaft 391 may comprise a first housing 397, which is a tube that houses the actuation element 398. In some embodiments, the clevis 360 is attached to the first housing 397. In the embodiment illustrated in FIGS. 3-8, the clevis 360 is attached to the first housing 397 via a retention pin 364, as shown in FIGS. 5 and 6. In other embodiments, the clevis 360 may be attached to the first housing 397 or to some other part of the shaft 391 by another fastening mechanism, such as mechanical fasteners, adhesives, welding, friction fitting, etc. The shaft 391 may also comprise additional housing structures beyond the first housing 397, such as the second housing 392 illustrated in FIG. 6. The second housing 392 is a tube that surrounds housing 397 and forms an outer wall of a portion of the shaft 391 (the second housing 392 is omitted from view in FIGS. 3 and 5). As would be familiar to those of ordinary skill in the art, additional structures could be included in the shaft 391 besides the first and second housings 397 and 392. Moreover, in some embodiments the second housing 397 may be omitted and the first housing 397 (or some other component) may act as the outer wall of the shaft 391. In FIGS. 3, 5, and 6, only a most distal portion of the shaft 391 is illustrated, but it should be understood that the shaft 391 may include other portions, which may include, for example, joints (such as the joints 296). Further, the jaw mechanism and clevis 360 may be coupled to the shaft 291 via a wrist mechanism (not shown in the embodiment of FIGS. 3-6) in various embodiments and the wrist mechanism may be configured to articulate relative to the shaft in pitch and/or yaw. A force transmission system, such as the force transmission system 292 described above, can be coupled at a proximal end portion of the shaft 391. The shaft 391 (shown in FIG. 6) may house various components, as described above with respect to the shaft 291. In particular, the shaft 391 houses one or more actuation elements, including the actuation element 398. The actuation elements (including the actuation element 398 (see FIGS. 6 and 8)) comprises push/pull cables, rods, or other devices for transferring motion/forces/torques received from drive inputs to the end effector 393 and/or other parts of the instrument (such as joints) to actuate functions of the instrument and/or drive other degrees of freedom of motion. For example, the actuation elements may be driven to translate along a longitudinal axis thereof, rotate about the longitudinal axis, or both, with the motion of the actuation elements driving motion of a corresponding part of the end effector 393 and/or joints. In embodiments in which one or more joints are included in the shaft 391, the actuation elements extending through such joints may be flexible in one or more dimensions to allow for the degrees of freedom of motion of the joint(s). In embodiments in which joints are included in the shaft 391, articulation of the joints may result in the shaft 391 and/or the actuation element 398 having different portions with different orientations relative to one another—e.g., a most distal portion of the shaft 391 and/or actuation element 398 may be oriented differently than a more proximal portion. Accordingly, references herein to the longitudinal axis of the actuation element 398 or longitudinal axis of the shaft 391 should be understood as referring to a longitudinal axis of a most distal portion of the shaft 391 or actuation element 398, which is a portion that extends between the end effector 393 and the joint(s) (if any), unless otherwise noted or implied by the context. Various shafts, actuation elements, joints, and other associated components (e.g., coil pipes to house a push/pull cable, shielding and insulation elements, etc.), are familiar to those of ordinary skill in the art, and thus detailed description of these components is omitted herein.

The jaw mechanism 350 comprises two jaw members 351, 352 (see FIG. 3), which are also referred to herein as a first jaw member 351 and a second jaw member 352. The jaw members 351, 352 comprise a distal working portion and a proximal end portion, with the proximal end portion being coupled to the clevis 360. The jaw members 351, 352 are arranged in opposition to one another such that pivoting of (at least) one of the jaw members 351, 352 relative to the clevis 360 causes the distal working portions of the jaw members 351, 352 to close together in a pinching motion. In particular, as shown in FIGS. 3 and 5, the first jaw member 351 has a proximal end portion 355 that is pivotably coupled to the clevis 360 by pivot connectors 361 which engage holes or recesses 363 in the clevis 360 and allow the first jaw member 351 to pivot relative to the clevis 360 about an axis 341. The axis 341 is perpendicular to the longitudinal axis of the shaft 391 and the longitudinal axis of the first jaw member 351, and may be referred to herein as a “pivot axis.” Pivoting of the first jaw member 351 about the pivot axis 341 causes the distal end of the first jaw member 351 to move toward or away from the second jaw member 352 to move the end of the first jaw member 351 along an arc as indicated by the arrows 343 in FIG. 3. FIGS. 3-6 illustrate the jaw mechanism 350 in an open position, in which the first jaw member 351 is angled away from the second jaw member 352. In a closed position (not illustrated) of the jaw mechanism 350, the jaw members 351, 352 may be positioned with a relatively small gap (or no gap) between the jaw members 351, 352.

The first jaw member 351 is driven to pivot about the pivot axis 341 by the actuation element 398 (see FIGS. 6 and 8). Specifically, in the embodiment illustrated in FIGS. 3-8, the actuation element 398 is drivable to translate along a longitudinal axis thereof (in a direction as indicated by the line 395 in FIG. 5-8). The translation of the actuation element 398 is converted into pivoting motion of the first jaw member 351 via a pin and slot engagement. As best seen in FIGS. 5-7, the actuation element 398 is coupled to the first jaw member 351 by a coupling mechanism 375 comprising a rotational coupling 380 and a closure block 370. As shown in FIG. 8, a proximal end portion 384 of the rotational coupling 380 is fixedly attached to the actuation element 398 such that translation of the actuation element 398 relative to the clevis 360 along the longitudinal axis of the actuation element 398 (parallel to line 395) causes the rotational coupling 380 to translate along the same direction. Furthermore, the closure block 370 is attached to the rotational coupling 380 such that the closure block 370 is constrained to translate with the rotational coupling 380. In particular, as shown in FIGS. 6 and 7, the closure block 370 has a roughly U-shaped cross-section with a central channel that receives the distal end 385 of the rotational coupling 380. The closure block 370 also comprises flanges 372 that extend into the channel to engage a complementary groove 381 in the outer surface of the rotational coupling 380, as shown in FIG. 7. The flanges 372 and groove 381 constrain the closure block 370 to translate with the rotational coupling 380, while also allowing relative rotation between the closure block 370 and the rotational coupling 380. Thus, translational movement of the actuation element 398 along its longitudinal axis is converted into translational movement of the closure block 370 in the same direction.

The closure block 370 comprises pegs 371 extending laterally from the sides of the closure block 370 (see FIG. 7). As shown in FIG. 5, the pegs 371 are received in guide slots 357 in the proximal portion 355 of the first jaw member 351 such that the translation of the closure block 370 relative to the clevis 360 causes the pegs 371 to slide along the guide slots 357. The closure block 370 and the first jaw member 351 are constrained by the clevis 360 such that the movement of the pegs 371 along the guide slots 357 forces the first jaw member 351 to pivot relative to the clevis 360 about the pivots 361. Thus, translation of the actuation element 398 along its longitudinal axis is converted by the rotational coupling 380, the closure block 370, and the orientation of the guide slots 357 into pivoting motion of the first jaw member 351. Thus, opening and closing of the jaw mechanism 350 is effectuated by translating the actuation element 398 to cause the first jaw member 351 to pivot between open and closed positions.

The jaw mechanism 350 is also capable of changing between multiple functional configurations to perform multiple different functions, as described above with respect to the end effector 293. The jaw mechanism 350 changes between these configurations by changing a pose of at least one of the jaw members 351, 352 and thereby moving different functional features of that jaw member 351, 352 into the active position. In the embodiment illustrated in FIGS. 3-8, it is the pose of the second jaw member 352 that is changeable to reconfigure the jaw mechanism 350, but in other embodiments the pose of the first jaw member 351 could be changeable to reconfigure the jaw mechanism 350 to perform a different function, in addition to or in lieu of changing the pose of the second jaw member 352. The changing of the pose of one of the jaw members 351, 352 may comprise rotating the jaw member 351, 352 and/or translating the jaw member 351, 352 between at least two different poses such that different functional features are brought into the active position in the respective poses.

For example, in the embodiment illustrated in FIGS. 3-8 the jaw mechanism 350 has a first configuration for performing a bipolar electrical sealing function (see FIG. 4A) and a second configuration for performing a bipolar electrical cutting function (see FIG. 4B), and the jaw mechanism 350 may be changed between these two configurations. In this embodiment, the jaw mechanism 350 changes between these two configurations by rotating the second jaw member 352 about a roll axis 342 of the second jaw member 352 (see FIG. 3) between a first orientation in which the electrode 354a is in the active position, as shown in FIG. 4A, and a second orientation in which the electrode 354b is in the active position, as shown in FIG. 4B. The electrodes 354a and 354b are examples of functional features of the second jaw member 352 and are described in greater detail below. Rotation of the second jaw member 352 about the roll axis 342 is indicated in FIG. 3 by the arrows 344. The roll axis 342 may be (approximately) parallel to a longitudinal axis of the second jaw member 352 and a longitudinal axis of the actuation element 398. Rotation of the second jaw member 352 about the roll axis 342 may also be referred to herein as rotation of the second jaw member 352 in a rotation degree of freedom of motion. The second jaw member 352 comprises a coupling portion 376 (see FIGS. 5-6) that rotatably couples the second jaw member 352 to the clevis 360. The coupling portion 376 comprises a groove 356 on a lateral exterior surface that engages a retention pin 362, which is secured to the clevis 360 (see FIGS. 5 and 6). As shown in FIG. 6, the parts of the coupling portion 376 that form the side walls or shoulders of the groove 356 engage with the retention pin 362 to prevent the second jaw member 352 from translating relative to the clevis 360 along the longitudinal axis of the second jaw member 352 while allowing rotation of the second jaw member 352 about the longitudinal axis.

The second jaw member 352 is driven to rotate about the roll axis 342 by one of the actuation elements. Specifically, in the embodiment illustrated in FIGS. 3-8 the actuation element that drives rotation of the second jaw member 352 is the same actuation element that drives pivoting of the first jaw member 351, namely the actuation element 398. As shown in FIGS. 5 and 6, the actuation element 398 is coupled to the second jaw member 352 by the rotational coupling 380. As noted above, the rotational coupling 380 has a proximal end portion 384 that is fixedly attached to the actuation element 398 (see FIGS. 6 and 8). The coupling between the rotational coupling 380 and the actuation element 398 is such that the rotational coupling 380 rotates with the actuation element 398 (in addition to translating with the actuation element 398, as already described above). The rotational coupling 380 also has a distal end portion 385 (see FIG. 8) that is coupled to a proximal end portion 358 of the second jaw member 352 (see FIG. 6). The coupling between the rotational coupling 380 and the proximal end portion 358 of the second jaw member 352 is such that the second jaw member 352 is constrained to rotate with the rotational coupling 380 while the rotational coupling 380 is translatable relative to the second jaw member 352 along a direction approximately parallel to the longitudinal axes of the rotational coupling 380, the second jaw member 352, and the actuation element 398. More specifically, in some embodiments the distal end portion 385 of the rotational coupling 380 and the proximal end portion 358 of the second jaw member 352 have mating anti-rotational features that prevent them from rotating relative to each other. In the embodiment shown, the distal end portion 385 of the rotational coupling comprises a bore 382 that receives the proximal end portion 358 of the second jaw member 352 (see FIGS. 6 and 8). As shown in FIG. 8, an inner surface of the rotational coupling 380, i.e., the surface that faces into and defines a boundary of the bore 382, comprises ridges 383 that protrude radially inward from the inner surface and extending longitudinally along a direction parallel to the longitudinal axis of the rotational coupling 380. These ridges 383 engage with complementary grooves 359 in an outer surface of the proximal end portion 358 of the second jaw member 352 (see FIG. 5). The ridges 383 and grooves 359 interact (mate) and act as anti-rotational features to constrain the second jaw member 352 to rotate with the rotational coupling 380 (in other words prevent rotation of the second jaw member 352 relative to the rotational coupling 380) while allowing the rotational coupling 380 to translate relative to the second jaw member 352. Thus, rotational movement of actuation element 398 is converted by the rotational coupling 380 into rotation of the second jaw member 352.

As would be appreciated by those of ordinary skill in the art, other mechanisms and configurations could be used to constrain the second jaw member 352 to rotate with the rotational coupling 380 while allowing the rotational coupling 380 to translate relative to the second jaw member 352. For example, in some embodiments, instead of ridges 383 being provided in the rotational coupling 380 and complementary grooves 359 being provided in the second jaw member 352, the reverse arrangement is used—i.e., ridges are provided protruding from an outer surface of the proximal end portion 358 of the second jaw member 352 and grooves are provided in the inner surface of the rotational coupling 380. Further, other numbers and/or arrangements around the periphery of such ridges and bores could be used without departing from the scope of the present disclosure. In addition, those having ordinary skill in the art would appreciate that other anti-rotational features than the grooves and ridges can be provided to achieve the desired constrained rotational movement of the second jaw member with the rotational coupling 380.

As another example, rather than the rotational coupling 380 comprising a bore 382 and the proximal end portion 358 of the second jaw member 352 being received inside the rotational coupling 380, in some embodiments the proximal end portion 358 of the second jaw member 352 comprises a bore and the distal end portion 385 of the rotational coupling 380 is received inside the second jaw member 352. In yet another embodiment, a ball-spline mechanism can be used to couple the second jaw member 352 to the rotational coupling.

As noted above, the rotational coupling 380 is coupled to the closure block 370 such that the rotational coupling 380 is rotatable relative to the closure block 370. Thus, rotation of the actuation element 398 to rotate the second jaw member 352 about the roll axis 342 does not affect the closure block 370, and hence does not affect a position/orientation of the second jaw member 352. Similarly, the rotational coupling 380 is coupled to the second jaw member 352 such that the rotational coupling 380 is translatable relative to the second jaw member 352. Thus, translation of the actuation element 398 to pivot the first jaw member 351 about axis 341 does not affect a position/orientation of the second jaw member 352. In other words, the coupling mechanism 375 that couples the actuation element 398 to the first and second jaw members 351, 352 allows the same actuation element 398 to independently actuate the first jaw member 351 to pivot and the second jaw member 352 to rotate.

As described above, in the embodiment illustrated in FIGS. 3-8 the same actuation element 398 independently drives rotation of the second jaw member and pivoting of the first jaw member 351. However, in other embodiments it is contemplated to use two separate actuation elements to drive the rotation of the second jaw member 352 and the pivoting of the first jaw member 351. In such embodiments, the rotational coupling 380 could be omitted, a first actuation element could be coupled to a mechanism for driving pivoting motion of the first jaw member 351 (such as the closure block 370), and a second actuation element could be coupled (directly or via an intermediary) to the second jaw member 352. As described above, in some embodiments the rotation of the second jaw member 352 is driven by rotation of an actuation element (e.g., the actuation element 398) and the pivoting of the first jaw member 351 is driven by translation of an actuation element (e.g., the actuation element 398), but in other embodiments other forms of motion of the actuation element(s) may be converted into the pivoting and/or rotation of the first and second jaw members 351, 352. For example, in some embodiments, rotational motion of an actuation element may be converted into pivoting motion of the first jaw member 351 by a motion conversion device such as a rack-and-pinion, screw drive, gears, etc. As another example, in some embodiments translational motion of an actuation element may be converted into rotational motion of the second jaw member 352 by a motion conversion device, such as a rack-and-pinion.

As noted above, in the embodiment illustrated in FIGS. 3-8 the end effector 393 is configured to perform a bipolar electrical sealing function and a bipolar electrical cutting function. Accordingly, the jaw members 351, 352 each comprise electrodes that are arranged to transmit electrical power through an object grasped by the jaw mechanism 350. More specifically, the first jaw member 351 comprises an electrode 353 (see FIG. 3) arranged on a bottom side of the first jaw member 351 facing the second jaw member 352. The second jaw member 352 comprises two electrodes 354a and 354b. In FIG. 3, the electrode 351 is physically and electrical separate from and coupled to a main body of the first jaw member 351, while the electrodes 354a and 354b are integral with one another and with a main body of the second jaw member 352. In other words, in the illustrated embodiment the electrode 351 comprises a conductive body that is coupled to the rest of the first jaw member 351 (which may comprise another conductive body or a non-conductive body), while the jaw member 352 comprises a single body of conductive material with the electrodes 354a and 354b being formed from different surface features of the second jaw member 352. In other embodiments, the electrode 351 could be integral with the rest of the first jaw member 351, and/or the electrodes 354a and 354b could be physically and/or electrically separate from one another and/or from a main body of the second jaw member 352. Thus, references herein to an “electrode” or an “electrode portion” should be understood as broadly referring to any part of a member of an end effector that is configured to function as an electrode, regardless of whether that part happens to be integral with the rest of the member or is a separate part coupled to the rest of the member.

The second jaw member 352 is rotatable about the roll axis 342 such that either one of the electrodes 354a and 354b can be brought into an active position, which is a position in which the electrode 354a or 354b is opposite from and facing the electrode 353. When an object, such as a blood vessel, is positioned between the jaw members 351, 352 and the jaw mechanism is closed (moved to the closed position), the electrode 353 and whichever of the electrodes 354a and 354b is in the active position may contact opposite sides of the object. This is referred to herein as the object being grasped by the jaw mechanism 350 or grasped between the jaw members 351, 352. Electrical power may then be supplied to the electrodes 353, and 353a or 354b, causing electricity to be transmitted through the grasped object. The electrical power transmitted through the grasped object causes sealing or cutting to occur in a portion of the object, depending on which electrode 354a or 354b is in the active position and the mode of electrical power that is applied, as described in greater detail below.

The electrode 354a is configured for a bipolar sealing function, while the electrode 354b is configured for a bipolar cutting function. The electrodes 354a and 354b are thus examples of functional features configured for performing particular functions. The electrode 354a is configured for bipolar sealing by virtue of having a contact surface for contacting the grasped object that is relatively broad in a lateral dimension (i.e., the width w₁ as illustrated in FIG. 4A), or in other words has a relatively large surface area, which may be particularly suitable for performing bipolar sealing, while the electrode 354b is configured for bipolar cutting by virtue of having a contact surface that is relatively narrow in the lateral dimension (i.e., the width w₂ as illustrated in FIG. 4B, which is less than w₁) or in other words has a relatively small surface area, which may be particularly suitable for performing bipolar cutting. The relatively broad contact surface of the electrode 354a tends to dissipate the electrical power over a relatively large area of the grasped object, which can be beneficial for a sealing function. On the other hand, the relatively narrow contact surface of the electrode 354b tends to concentrate the electrical power over a relatively small area of the grasped object, which can be beneficial for a cutting function. Sealing (coagulation) and cutting (vaporization) of tissue can occur as a result of heat generated by the electricity passing through the tissue. When relatively high amounts of heat are generated quickly, vaporization of the tissue occurs, whereas relatively moderate amounts of heat result in coagulation. Thus, the sealing and cutting functions may be effectuated by controlling the amount of heat generated by the electricity.

One way that the amount of heat that is generated is controlled is by controlling the mode of the electrical power, such as the voltage, current, frequency, and duty cycle at which the electrical power is applied. However, the area over which the electrical power is applied also affects the amount of heat generated. Because the electrode 354a dissipates the electrical energy over a relatively wider area by virtue of the relatively broad contact surface, the amount of heat generated by the electrical energy at any given point is relatively lower (all other things being equal), thus reducing the likelihood of inadvertent vaporization of tissue or collateral tissue damage. Moreover, the relatively broader contact surface may result in a wider sealing area (i.e., a wider region of coagulation), which may result in a surer seal (e.g., less likelihood of the vessel leaking or reopening). On the other hand, because the electrode 354b concentrates the electrical power in a relatively small area by virtue of the relatively narrow contact surface, the heat generated by the electrical power is relatively higher (all other things being equal), thus facilitating the vaporization of the tissue. Moreover, the relatively narrow contact surface results in a relatively narrow region of the tissue being vaporized, thus reducing the amount of tissue that is destroyed by the cutting function, or in other words allowing for more precise and narrow cuts to be performed.

It should be noted that sealing and cutting do not necessarily require broad and narrow electrodes, respectively. Instead, sealing can be performed with relatively narrow electrodes and cutting can be performed with relatively broad electrodes, if the appropriate mode of electrical power is delivered under the appropriate circumstances. However, all other things being equal, an electrode with a broader contact surface may be better able to perform sealing (e.g., may generate a surer seal, more efficiently, and/or with less collateral tissue damage), and an electrode with a narrower contact surface may be better able to perform cutting (e.g., may generate a narrower cut, more efficiently, and/or with less collateral tissue damage). Thus, references herein to the electrodes 354a and 354b being configured to perform sealing or cutting functions, respectively, should not be misunderstood as implying that the electrodes 354a and 354b can perform only sealing or only cutting functions. Similarly, references herein to the electrodes 354a and 354b being configured to perform “different” functions should not be misunderstood as implying that the electrodes 354a and 354b perform mutually exclusive functions or cannot perform the same function as one another. Rather, it should be understood that such references mean that the electrodes 354a and 354b each have something different about their respective configurations (e.g., their shapes, sizes, surface features, material, etc.) that adapts the electrodes to be more suitable for a particular function, without necessarily precluding their ability to perform other functions. Thus, for example, two electrodes may be considered as being configured to perform different functions if one of the electrodes is relatively wider in a lateral dimension than the other.

An insulation layer 348 is provided between the electrode 353 and a top portion 349 of the first jaw member 351. The insulation layer 348 comprises a non-conductive material (i.e., a material with negligible electrical conductivity, such as a plastic, a polymer, rubber, ceramic, glass, etc.), and insulates the electrode 353 from the top portion 349 to prevent or reduce leakage current or unintentional electrical discharge to the top portion 349 in embodiments in which the top portion 349 is conductive. The first jaw member 351 may also include non-conductive standoffs 347 to prevent the electrode 353 from directly contacting the second jaw member 352 and forming a short circuit.

The electrode 353 of the first jaw member 351 is electrically coupled to an electrical energy transmission line 345 (see FIG. 6), which may extend through or along the shaft 391 to electrically couple with a power source. The power source (not shown) may be, for example, an electrical input interface of an input portion of an instrument that is to couple with and receive electrical power from an electrical output interface that is separate from the instrument. For example, the electrical output interface may be part of a manipulator, a connector of a power cord coupled to a power supply unit (e.g., an electrosurgical unit (“ESU”) or electrosurgical generator), or the like. As another example, the power source may be a battery or other power supply device that is part of the instrument. The electrodes 354a and 354b are coupled to a second electrical energy transmission line (not illustrated), which is also electrically coupled to the power source. The electrode 353 may be coupled to a “hot” node of the power source, which carries an electrical potential relative to a reference ground, while the electrodes 354a and 354b may be coupled to a neutral/return or ground reference node of the power source, or vice-versa. Thus, when an object is grasped by the jaw mechanism 350 and electrical energy is supplied to the electrodes 353, 345a, and 345b, an electrical potential difference (voltage difference) is created between the electrode 353 and the electrode 345a or 345b. The material grasped by the jaw mechanism 350 completes a circuit and thus current flows between the electrode 353 and the electrode 345a or 345b passing though the grasped object. In some embodiments, the electrical energy transmission line 345 extends from the electrode 353, through or along the first jaw member 351 (e.g., via channels, not visible), through or along the clevis 360 (e.g., via channels, not visible), and then through or along the shaft 391. In the embodiment illustrated in FIGS. 3-8, the electrodes 354a and 354b are both parts of the same unitary body that forms the second jaw member 352. In other embodiments, the electrodes 354a and 354b are separately formed parts that are coupled to the second jaw member 352.

In some embodiments, the electrical energy transmission line to which the electrodes 354a and 354b are coupled may be formed from another component of the instrument that also serves another purpose. For example, any one or more of the actuation element 398 (or some other actuation element), the first housing 397 that houses the actuation element 398, and/or another conductive component running through the shaft 391 may be used as part of the electrical energy transmission line for the electrodes 354a and 354b. Furthermore, in some embodiments, various components of the end effector 393 comprise electrically conductive materials and may form a portion of the electrical energy transmission line of the electrodes 354a and 354b, such as for example, any one or more of the closure block 370, the rotational coupling 380, the retention pin 362, and/or the clevis 360. Thus, existing components that have other functions may be used to form part (or all) of the electrical energy transmission line for the electrodes 354a and 345b, and a separate electrical energy transmission line does not have to be provided. However, it should be understood that a separate electrical energy transmission line could be provided for the electrodes 354a and 354b, if desired.

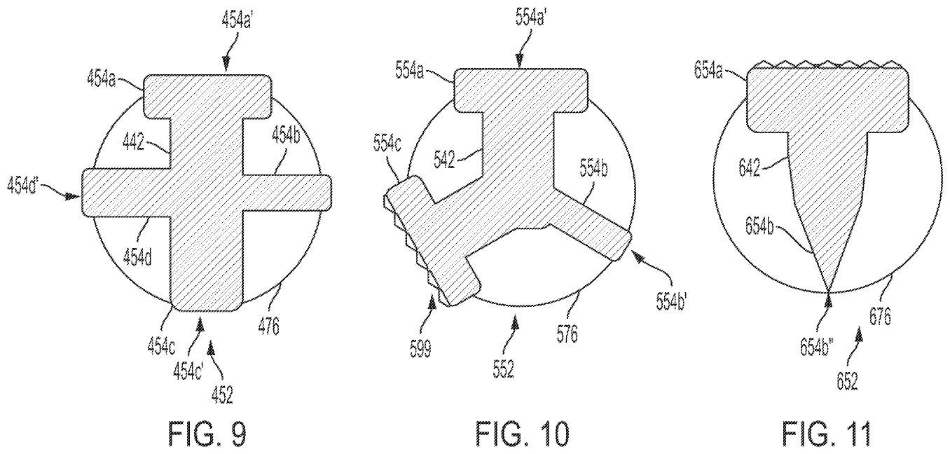

In the embodiment of FIGS. 3-8, the second jaw member 352 has two functional features, i.e., the electrodes 345a and 345b. However, embodiments of the present disclosure are not limited to multifunction members having these specific functional features, or to having just two functional features. In particular, a multifunction member 252 of the end effector 293 may comprise any number of functional features to perform any number of functions, which number may be selected based on a variety of factors, such as, for example, the space available in view of the size of the functional features and end effector, and the particular procedures desired to be performed. FIGS. 9-11 illustrate various embodiments of multifunction jaw members 452, 552, and 652 having a variety of different numbers and types of functional features. The multifunction jaw members 452, 552, and 652 could be used, for example, in the end effector 393 to replace the jaw member 352. In some embodiments, the multifunction jaw members 452, 552, and 652 may be similar to the jaw member 352 except that the multifunction jaw members 452, 552, and 652 may have differently configured functional features. Moreover, in the embodiments illustrated in FIGS. 3-11, the functional features are illustrated as being parts of the same monolithic structure, but embodiments of the present disclosure are not limited to multifunction members with such configurations. In some embodiments, one, some, or all of the functional features (for example, one or more of the electrodes 345a and 345b) may be physically distinct from and supported by a main body of the multifunction jaw member.

FIG. 9 is a cross-section of a multifunction jaw member 452 taken in a plane transverse to the longitudinal axis of the jaw member 452. The multifunction jaw member 452 comprises four functional features 454a to 454d. The functional features 454a to 454d are distributed around a periphery of the multifunction jaw member 452, such that rotation of the multifunction jaw member 452 about an axis 442 selectively brings the functional features 454a to 454d into an active position (e.g., a position at the top of the multifunction jaw member 452 in FIG. 9, although the active position could be any desired angular position around the axis 442). The axis 442 extends in a proximal-distal direction and may be approximately parallel to a longitudinal axis of the multifunction jaw member 452. In FIG. 9, the functional features 454a to 454d are electrodes for performing electrosurgical functions, including bipolar electrical sealing and bipolar electrical cutting. Each of the functional features 454a to 454d has a different configuration. Specifically, each of the functional features 454a to 454d has a contact surface 454a′ to 454d′ with a different width in a lateral dimension (dimension generally perpendicular to the longitudinal axis), or in other words a different surface area. The functional feature 454a has the broadest contact surface 454′ and is configured for performing a sealing function. The functional feature 454b has the narrowest contact surface 454b′ and is configured for performing a cutting function. The functional features 454c and 454d have contact surfaces 454c′ and 454d′ with intermediate widths and can be used for sealing or cutting, depending on a desired effect. For example, if a relatively narrower seal is desired than can be achieved with the functional feature 454a, then one of the functional features 454c and 454d could be used to achieve a narrower seal. The multifunction jaw member 452 also comprises a coupling portion 476 for rotatably coupling the multifunction jaw member 452 to another portion of the end effector (such as to the clevis 360), which may be similar to the coupling portion 376 described above. The multifunction jaw member 452 also comprises a proximal portion (not visible) for coupling with a coupling mechanism (such as the coupling mechanism 375) to drive rotation of the multifunction jaw member 452. The proximal portion may be similar to the proximal portion 358 described above.

FIG. 10 comprises a cross-section of another embodiment of a multifunction jaw member 552. The multifunction jaw member 552 comprises three functional features 554a to 554c. The functional features 554a to 554c are distributed around a periphery of the multifunction jaw member 552, such that rotation of the multifunction jaw member 552 about an axis 542 selectively brings the functional features 554a to 554c into an active position (e.g., a position at the top of the multifunction jaw member 552 in FIG. 10 though other positions are contemplated as within the scope of the disclosure). The axis 542 is approximately parallel to a longitudinal axis of the multifunction jaw member 552. In FIG. 10, the functional features 554a and 554b are electrodes for bipolar electrical sealing and bipolar electrical cutting (similar to the electrodes 354a and 354b), while the functional feature 554c is for performing a grasping function. Specifically, the functional feature 554c is adapted to perform a grasping function by virtue of having friction enhancing features 599 on a contact surface thereof. Friction enhancing features 599 may include, for example, a series of protrusions, grooves, knurling, pumps, or other similar surface features. Although electrodes may also be used to grasp objects, this is not their primary function and they may be less effective at securely grasping objects, such as tissue, due to slippage for the grasped objects. Moreover, friction-enhancing surface features offering sufficient friction for grasping may not allow the electrode to perform as desired for sealing and/or cutting. However, the functional feature 554c may be more effective at securely grasping objects, due to the friction enhancing features 599. The functional features 554a and 554b may have contact surfaces 554a′ and 554b′ with different widths in a lateral dimension, similar to the functional features 454a and 454b described above. The multifunction jaw member 552 also comprises a coupling portion 576 for rotatably coupling the multifunction jaw member 552 to an end effector housing (such as the clevis 360), which may be similar to the coupling portion 376 described above. The multifunction jaw member 552 also comprises a proximal portion (not visible) for coupling with a coupling mechanism (such as the coupling mechanism 375) to drive rotation of the multifunction jaw member 552. The proximal portion may be similar to the proximal portion 358 described above.

FIG. 11 comprises a cross-section of yet another embodiment of a multifunction jaw member 652. The multifunction jaw member 652 comprises two functional features 654a and 654b. The functional features 654a and 654b are distributed around a periphery of the multifunction jaw member 652, such that rotation of the multifunction jaw member 652 about an axis 642 selectively brings the functional features 654a and 654b into an active position (e.g., a position at the top of the multifunction jaw member 652 in FIG. 11). The axis 642 is approximately parallel to a longitudinal axis of the multifunction jaw member 652. In FIG. 10, the functional features 654a is for performing a grasping function, similar to the functional feature 554c described above, and the functional feature 654b is for performing a mechanical cutting function. Specifically, the functional feature 654b is adapted for mechanical cutting by virtue of having a sharp edge 654b″. If tissue is positioned between the multifunction jaw member 652 and an opposing jaw member (such as the first jaw member 351), with the functional feature 654b in the active position, and the jaw mechanism is closed, then the grasped tissue will be squeezed between the functional feature 654b and the opposing jaw member, resulting in the sharp edge of the functional feature 654b cutting through the grasped tissue. The multifunction jaw member 652 also comprises a coupling portion 576 for rotatably coupling the multifunction jaw member 652 to an end effector housing (such as the clevis 360), which may be similar to the coupling portion 376 described above. The multifunction jaw member 652 also comprises a proximal portion (not visible) for coupling with a coupling mechanism (such as the coupling mechanism 375) to drive rotation of the multifunction jaw member 652. The proximal portion may be similar to the proximal portion 358 described above.

The above-described embodiments of the multifunction jaw members 352, 452, 552, and 652 are provided as illustrative of how various types and numbers of functional features can be included as parts of a jaw member. However, these examples are not limiting. For example, in some embodiments functional features other than those illustrated are used, in addition to or in lieu of the illustrated functional features, such as: a stapler for firing staples, an anvil for closing staples, a lens of a camera system, an inlet/outlet of a flow removal/delivery system such as a vacuum suction system or gas delivery system, a sensing device (e.g., to sense temperature, electricity, pressure, etc.), a light or other electromagnetic energy delivery feature, or any other functional feature. Moreover, various embodiments disclosed herein include combinations of functional features described herein, including any combination of two or more of the functional features.

Moreover, although some of the embodiments described above focus on jaw mechanisms, embodiments of end effectors disclosed herein are not limited to jaw mechanisms. For example, in some embodiments, an end effector comprises a multifunction member and another member, but the other member is not arranged in opposition to the multifunction member to form a jaw mechanism. In some embodiments, an end effector comprises a multifunction member without necessarily having any other member, such as an opposing jaw member. For example, in some embodiments an end effector comprises a single member with multiple monopolar electrodes or bipolar electrodes that are disposed on (or are part of) the member; the member may be shaped as, for example, a spatula, a hook, or other similar non-jawed end effector.

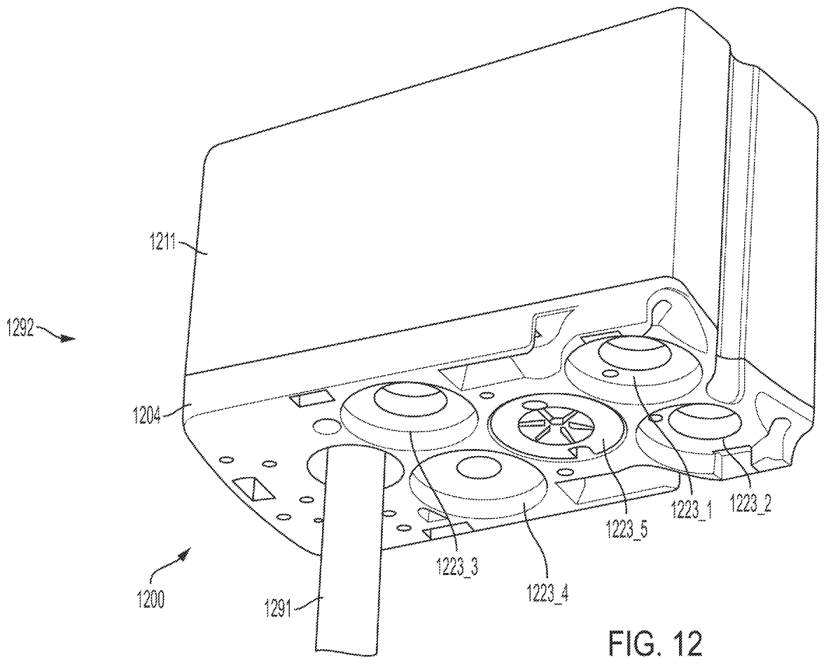

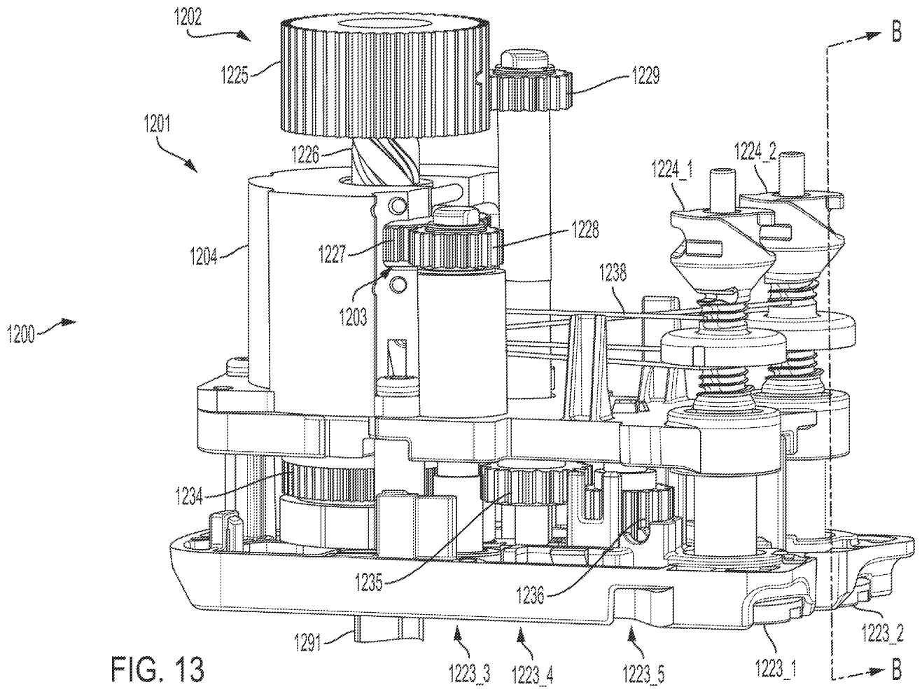

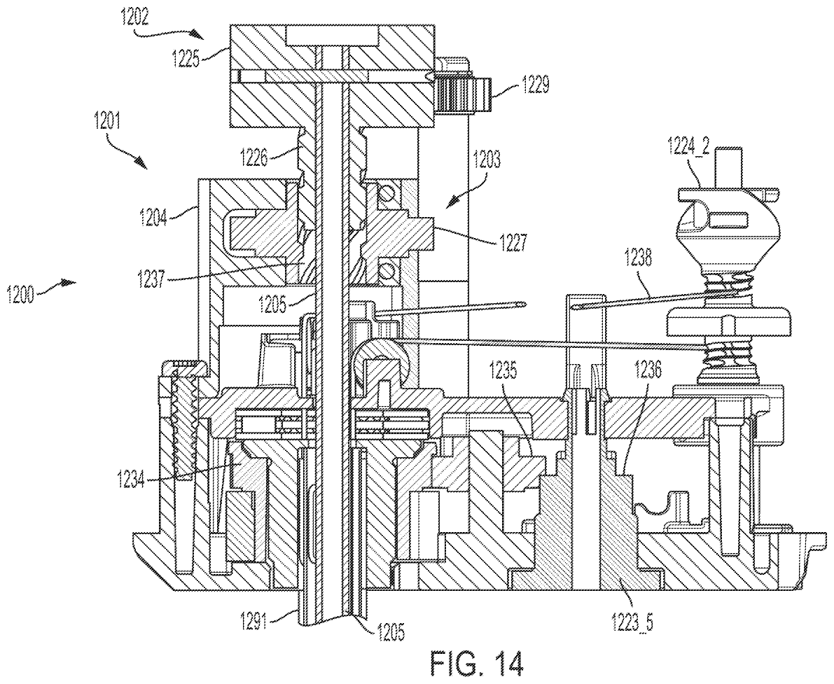

As described above with respect to the end effector 393, in some embodiments a single actuation element (e.g., actuation element 398) can be used to actuate both the open/close motion of a first jaw member and a rotation of a second jaw member to change a functional configuration of the end effector. Such an actuation element may be independently driven both in translation (e.g., to actuate the first jaw member) and in rotation (e.g., to actuate the second jaw member). Accordingly, in such embodiments a force transmission system of the instrument may be configured to independently impart translation and rotation to a given actuation element, which is coupled directly or indirectly to the end effector. FIGS. 12-14 illustrate one embodiment of such a force transmission system 1200. FIG. 12 comprises a perspective view that illustrates exterior components of the force transmission system 1200. FIG. 13 comprises a perspective view that illustrates the force transmission system 1200 with the force transmission housing 1211 removed to reveal internal force conversion components 1292 of the force transmission system 1200. FIG. 14 comprises a cross-section of the force transmission system 1200 with the section taken along B in FIG. 13.

As shown in FIG. 12, a force transmission system 1200 comprises a housing 1211 and a chassis 1204 fitted with a number of drive inputs 1223. The housing 1211 houses and the chassis 1204 supports various components of the force transmission system 1200, such as force conversion components 1292, which are described in greater detail below. The chassis 1204 may be configured to be mountable to an interface of a manipulator. The drive inputs 1223 may be used as the drive inputs 223 described above. In FIGS. 12-14, five drive inputs 1223 are illustrated, namely drive inputs 1223_1 through 1223_5, but in other embodiments any number of drive inputs 1223 can be used. The drive inputs 1223 are configured to interface with complementary drive outputs of a manipulator (either directly, or via an intermediary such as a sterile adaptor), such that the drive outputs mechanically transfer driving forces, torques, and/or motion to the drive inputs 1223. For example, the drive inputs 1223 may be rotary discs.

The force transmission system 1200 further comprises force conversion components 1292, which comprise a variety of force conversion mechanisms coupled between the drive inputs 1223 and actuation elements (e.g., actuation element 1205, cables 1238), as shown in FIGS. 13 and 14. These force conversion components 1292 transfer mechanical inputs (force, torque, motion) imparted to the drive inputs 1223 to the actuation elements, which extend through the shaft 1291 of the instrument (partially shown in the views of FIGS. 12-14) to distal portions of the instrument, such as one or more of an end effector and a joint. The forces and motion imparted to the actuation elements drive actuation of functions of the instrument and/or movement of the instrument through one or more degrees of freedom, e.g., articulating a joint, translating a component of the end effector, opening/closing jaws of the end effector, etc.

In particular, the force conversion components 1292 comprise a mechanism 1201 to drive a given actuation element 1205 to independently translate and rotate. The actuation element 1205 (see FIG. 14) may be any element capable of being driven to transmit both linear force/translational motion and torque/rotational motion. For example, the actuation element 1205 may be a rod as shown in FIG. 14, a push/pull cable (e.g., housed in a coil pipe), or any other element capable of transmitting linear and rotational forces/motion. In some embodiments, this actuation element 1205 may be coupled, directly or indirectly, to a jaw mechanism (such as the jaw mechanism 350) to drive independent actuation of both a pivoting motion of a first jaw member and rotational motion of a second jaw member about a longitudinal axis of the second jaw member. In some embodiments, one or more joints may be included in the shaft 1291 and at least a portion of the actuation element 1205 may be flexible in one or more dimensions to accommodate movement of the joint(s), or the actuation element 1205 may be coupled to a separate actuation element that is flexible in one or more dimensions and which passes through the joint(s). The actuation element 1205 may be used as, or may be coupled to, the actuation element 398 discussed above with reference to the embodiments of FIGS. 3-8.

The force conversion mechanism 1201 for driving translation and rotation of the actuation element 1205 comprises gears 1225 to 1229, which are coupled to and driven by drive inputs 1223_3 and 1223_4. Rotation of the actuation element 1205 relative to the shaft 1291 can be effectuated by rotating the drive input 1223_4. As shown in FIG. 13, the drive input 1223_4 is coupled to the gear 1229 such that the gear 1229 is constrained to rotate with the drive input 1223_4. The gear 1229 is engaged with the gear 1225. The gear 1225 is coupled to the actuation element 1205 such that the actuation element 1205 is constrained to translate and rotate with the gear 1225. Accordingly, rotation of the drive input 1223_4 causes rotation of the actuation element 1205 relative to the chassis 1204. The shaft 1291 may be held rotationally stationary relative to the chassis 1204 (for example, by the gear 1234 or by the shaft 1291 being fixedly coupled to the chassis 1204). Accordingly, the rotation of the actuation element 1205 relative to the chassis 1204 while the shaft 1291 is held rotationally stationary results in rotation of the actuation element 1205 relative to the shaft 1291. In other words, rotation of the drive input 1223_4 can drive rotation of the actuation element 1205 relative to the shaft 1291. Accordingly, in embodiments in which the transmission system 1200 is used to drive the end effector 393 described above and the actuation element 1205 is used as or coupled to the actuation element 398, rotation of the multi-function jaw member 352 may be effectuated by driving the drive input 1223_4 to rotate.

Translation of the actuation element 1205 along an axial direction relative to the shaft 1291 can be effectuated by driving the drive input 1223_3 to rotate while the drive input 1223_4 is held stationary. The translation of the actuation element 1205 along the axial direction is controlled by the interaction between the gears 1226 and 1237, specifically by relative rotation between the gears 1226 and 1237 (see FIG. 14). As shown in FIG. 14, the gear 1226 is an external screw/worm gear configured to be inserted into and engaged with the gear 1237, which is an internal screw/worm gear. Thus, rotation of the gears 1226 and 1237 relative to one another causes them to translate axially relative to one another. As shown in FIGS. 13 and 14, the gear 1226 is coaxial with and coupled to the gear 1225, with the gears 1225 and 1236 forming a gear assembly 1202 that rotates and translates together. Moreover, as shown in FIG. 14, the gear 1227 is coaxial with and coupled to a gear 1237, with the gears 1227 and 1237 forming a gear assembly 1203 that rotates and translates together. The gear assembly 1203 is coupled to the chassis 1204 such that the gear assembly 1203 is rotatable relative to the chassis 1204 but constrained to prevent translation of the gear assembly 1203 in an axial direction. Accordingly, relative rotation of the gears 1226 and 1237 causes the gear assembly 1202 to translate axially relative to the chassis 1204. Because the shaft 1291 is translationally fixed relative to the chassis 1204 and the actuation element 1205 is translationally fixed relative to the gear assembly 1202, the translation of the gear assembly 1202 axially relative to the chassis 1204 (due to the relative rotation of the gears 1226 and 1237) results in translation of the actuation element 1205 relative to the shaft 1291. The gears 1226 and 1237 are driven by the drive inputs 1223_3 and 1223_4. As shown in FIG. 13, the drive input 1223_3 is coupled to the gear 1228 such that the gear 1228 is constrained to rotate with the drive input 1223_3, and the gear 1228 is engaged with the gear 1227. Similarly, the drive input 1223_4 drives rotation of the gear 1225 (via gear 1229), which drives rotation of the gear 1226. Thus, relative rotation of the gears 1226 and 1237, and hence translation of the actuation element 1205, can be effectuated by driving the drive inputs 1223_3 and 1223_4 such that the gears 1226 and 1237 have different angular velocities-for example, by driving the drive input 1223_3 to rotate while holding the drive input 1223_4 stationary. Accordingly, in embodiments in which the transmission system 1200 is used to drive the end effector 393 described above and the actuation element 1205 is used as or coupled to the actuation element 398, pivoting of the jaw member 351 may be effectuated by driving the drive input 1223_3 to rotate while holding the drive input 1223_4 stationary.

If it is desired to prevent translation of the actuation element 1205 while the actuation element 1205 is being driven to rotate, then the drive input 1223_3 can be driven along with the drive input 1223_4 such that the gears 1226 and 1237 remain stationary relative to one another (i.e., have the same angular velocity). In some embodiments, the gears 1225, 1227, 1228, and 1229 are configured such that rotation of the drive inputs 1223_3 and 1223_4 at the same angular velocity results in rotation of the gears 1226 and 1237 at the same angular velocity. If it is desired to prevent rotation of the actuation element 1205 while the actuation element 1205 is being driven to translate, then the drive input 1223_4 can be held stationary while the drive input 1223_4 is rotated. Thus, the mechanism 1201 allows for the translation and rotation of the actuation element 1205 to be independently driven. On the other hand, if it is desired to simultaneously rotate and translate the actuation element 1205, then the drive input 1223_4 can be driven to rotate while also controlling the drive input 1223_3 such that the gears 1226 and 1237 have different respective angular velocities.

In some embodiments, the gear assembly 1203 is formed as a single unitary body, with the gears 1227 and 1237 being two integrally connected parts of the body, or in other words with the gears 1227 and 1237 being formed from inner and outer surfaces of the same body. In other embodiments, the gears 1227 and 1237 can be separate and distinct parts that are attached together to form the gear assembly 1203. In some embodiments, the gear assembly 1202 is formed as a single unitary body, with the gears 1225 and 1226 being integrally connected parts of the body, or in other words with the gears 1225 and 1226 being formed from two outer surfaces of the same body having different radii. In other embodiments, the gears 1225 and 1226 can be separate and distinct parts that are attached together to form the gear assembly 1202. Those having ordinary skill in the art would appreciate a variety of arrangements for the gear assemblies 1202 and 1203 that could be used without departing from the scope of the present disclosure.

The force transmission system 1200 may also comprise additional drive inputs 1223 besides those described above to drive other actions of the instrument, such as movement of joints or actuation of other functions. For example, in the embodiment illustrated in FIGS. 12-14, drive inputs 1223_1, 1223_2, and 1223_5 are included to drive motion of the instrument through three degrees of freedom of motion (i.e., yaw and pitch of the end effector, and roll of the instrument shaft) beyond those imparted by the manipulator to which the instrument is coupled. Other instrument may have more, fewer, or no degrees of freedom of motion. In embodiments in which additional drive inputs 1223 are included, the force transmission system 1200 may also include associated mechanisms, such as the gears 1234 to 1237 and capstans 1224_1 and 1224_2 described further below, to transmit the mechanical inputs from these additional drive inputs 1223 to actuation elements. It should be understood that more or fewer (including zero) such additional drive inputs 1223 and associated force transmission systems could be included in various embodiments, depending on the desired functions and degrees of freedom of motion of the instrument.

The drive input 1223_5 may control a “roll” degree of freedom of motion of the shaft of the instrument. As shown in FIGS. 13 and 14, drive input 1223_5 is coupled to the gear 1236 such that the gear 1236 is constrained to rotate with the drive input 1223_5. The gear 1236 is engaged with the gear 1235. As shown in FIG. 14, the gear 1235 is engaged with the gear 1234. The gear 1234 is coupled to the chassis 1204 such that the gear 1234 is rotatable relative to the chassis 1204 but constrained to prevent translation of the gear 1234 in an axial direction. As shown in FIG. 14, the gear 1234 is coupled to and coaxial with the shaft 1291, such that the shaft 1291 is constrained to rotate with the gear 1234. The shaft 1291 is rotatable relative to the chassis 1204. Thus, the shaft 1291 can be rotated relative to the chassis 1204 by driving rotation of the gear 1234 via the drive input 1223_5. Such rotation of the shaft 1291 relative to the chassis 1204 can, for example, cause the end effector (not shown in FIGS. 12-14) to rotate around a longitudinal axis of the shaft 1291 (i.e., a roll movement). If it is desired to rotate shaft 1291 without also causing movement of the first and second jaw members, then the gear assemblies 1202 and 1203 may be driven to rotate along with the gear 1234 (with the same angular velocities) during the roll operation.

In embodiments in which the shaft 1291 is rotatable relative to the chassis 1204 in a roll degree of freedom, the shaft 1291 may be controlled to remain stationary during an operation such that the actuation element 1205 rotates relative to the shaft 1291. Thus, in the illustrated embodiment, the drive input 1223_5 (and hence gear 1234) may be held rotationally stationary while the drive input 1223_4 is rotated during an operation to rotate the actuation element 1205. In other embodiments (not illustrated), the shaft 1291 may be permanently rotationally stationary relative to the chassis 1204.

The drive inputs 1223_1 and 1223_2 may control “yaw” and “pitch” degrees of freedom of motion of the end effector and/or a wrist of the instrument. As shown in FIGS. 13 and 14, capstans 1224_1 and 1224_2 are coupled to the drive inputs 1223_1 and 1223_2, respectively, such that the capstans 1224_1 and 1224_2 rotate with the drive inputs 1223_1 and 1223_2, respectively. The rotation of the capstans 1224_1 and 1224_2 winds and unwinds cables 1238 coupled to the capstans 1224_1 and 1224_2, thereby imparting driving forces/motion to the cables 1238. The cables 1238 are routed (e.g., via pulleys) to extend through the shaft 1291, as shown in FIG. 14, and transfer the driving forces/motion to one or more joints (not visible) to move the joint(s) through one or more degrees of freedom of motion. Capstans and associated cable drive systems, as well as joints and other devices driven by such cable drive systems, would be familiar to those of ordinary skill in the art, and thus are not described in greater detail herein.

As noted above, in some embodiments the end effector may comprise a monopolar electrode configured to perform a monopolar electrosurgical function, such as monopolar sealing or cutting. In general, a monopolar electrode is formed from an electrically conductive material, such as stainless steel or other metal, that is coupled to an electrical power source. The monopolar electrode is positioned near tissue that is to be cut or coagulated, and electricity is discharged from the electrode into the tissue to cause the cutting/coagulation. With monopolar devices, the electricity discharged from the electrode into the tissue is not returned to the power source via a return path in the instrument (e.g. via an opposing jaw member). Instead, the electricity flows from the electrodes into the patient's body, which forms the return path for the electrical discharge. A patient return electrode, which is separate from the surgical instrument, is placed in contact with the patient to complete a return circuit for the electricity discharged into the patient's body. In some cases, the monopolar electrode is shaped to have a relatively small tip so as to provide a relatively concentrated electrical discharge from the tip to the target tissue and to allow for increased precision. The electrode tip can have various shapes, such as a sphere, a cone, a wire loop, etc.

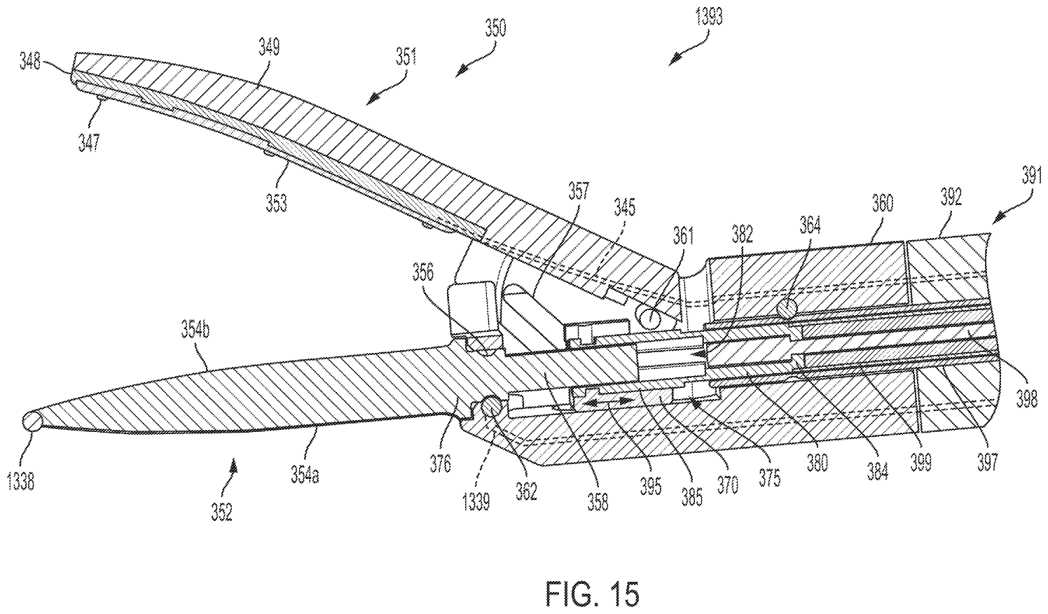

In some embodiments, a monopolar electrode is provided as one of the functional features of a rotatable multifunction member, such as the rotatable multifunction jaw member 352 or a rotatable multifunction member that is not part of a jaw mechanism. For example, FIG. 15 illustrates one embodiment of an end effector 1393 in which one or more monopolar electrodes are provided as part of the rotatable multifunction jaw member 352. The end effector 1393 may be similar to the end effector 393 described above, and components of the end effector 1393 are given the same reference numerals in FIG. 15 as similar components of the end effector 393 described above. Duplicative description of these similar components of the end effector 1393 is omitted.

As shown in FIG. 15, a distal tip of the multifunction jaw member 352 comprises a monopolar electrode 1338. In this embodiment, the multifunction jaw member 352 may be pointed at the target tissue and positioned such that the monopolar electrode 1338 is near or in contact with the target tissue, and then electrical power may be supplied to the jaw member 352, causing electricity to discharge from the monopolar electrode 1338 into the target tissue. Because the monopolar electrode 1338 is the closest part of the jaw member 352 to the target tissue in the above-described state, the electrical discharge from the jaw member 352 is concentrated primarily at and around the electrode 1338, thus providing a relatively intense and precise discharge. In FIG. 15, the monopolar electrode 1338 is shown as having a spherical shape, but this is non-limiting and other electrode shapes, as described above and as those of ordinary skill in the art would appreciate, are contemplated herein. In particular, any shape of electrode may be used as a monopolar electrode 1338. For example, a tip or end of the jaw member 352 as shown in FIGS. 3-4B may be used as the electrode 1338.

To supply electrical power to the multifunction jaw member 352 (and hence to the electrodes 1339, 354a, and 354b, which are integral parts of the jaw member 352 in the illustrated embodiment), an electrical power line 1339 may be coupled between one or more power sources (not illustrated) and the multifunction jaw member 352. The electrical power line 1339 may extend through the clevis 360, as shown schematically with dashed lines in FIG. 15. A rotatable coupling may be used between the electrical power line 1339 and the multifunction jaw member 352 to allow for the rotation of the multifunction jaw member 352. For example, in FIG. 15 the electrical power line 1339 is coupled to the retention pin 362, which is coupled to the multifunction jaw member 352 in the groove 356 and which allows for rotation of the multifunction jaw member 352. This embodiment of the rotatable coupling is non-limiting, and other rotatable couplings may be used to electrically couple the electrical power line 1339 and the multifunction jaw member 352. Rotatable electrical couplings would be known to those of ordinary skill in the art, and thus are not described in detail herein.

Alternatively, in some embodiments a sperate power supply line 1339 is not provided, and instead other components of the instrument, such as the rotational coupling 380 and actuation element 398, may be made from electrically conductive materials and may be used as part of the power line to couple the jaw member 352 to a power source, as described above.

In some embodiments, the electrodes 354a and 354b of the jaw member 352 may also be configured as monopolar electrodes in addition to or in lieu of providing the monopolar electrode 1338 on the jaw member 352. The electrodes 354a and 354b, being positioned along lateral side surfaces of the jaw member 352, may be used in a different manner than the electrode 1338. For example, instead of pointing the jaw member 352 at the target tissue, the target tissue may be positioned adjacent to the lateral sides of the jaw member (e.g., below the jaw member 352 or between the jaw members 351 and 352) and then electrical power may be supplied to the jaw member 352. In such a state, a relatively larger area of electrode 354a or 354b is near to the target tissue, as compared to when the electrode 1338 is used as described above, resulting in a relatively more diffuse discharge of electricity over a relatively broader area of tissue. In some embodiments the electrodes 354a and 354b may be switched between monopolar and bipolar electrical energy delivery states by changing how electricity is supplied to the end effector 1393. Specifically, by coupling the multifunction jaw member 352 to a power supply line of a monopolar power source while disconnecting the electrode 353 from electrical power (i.e., the electrode 353 is electrically isolated or left “floating”), the electrodes 354a and 354b and the electrode 1338 (if present) are configured to function as monopolar electrodes. The electrode 353 of the opposing jaw member 351 is disconnected from the power source when operating in monopolar mode in order to prevent the electricity discharged from the electrodes 1338, 354a, or 354b from using the electrode 353 as a return path. On the other hand, by connecting the electrode 353 to a power supply line of a bipolar power source and connecting the jaw member 352 to a return line of the bipolar power source (or vice-versa), the electrodes 354a and 354b may be reconfigured to function as bipolar electrodes (the electrode 1338 may be considered as a part of the electrode 354a in such a configuration).

To change the electrical connections to the electrodes 354a and 354b, the power line 1339 (or other electrical path that couples the jaw member 352 to the power source) may be coupled to an electrical switch (e.g., a transistor or other switching device), which is coupled to power lines of one or more power sources. Another electrical switch may also be used to selectively connect and disconnect the power line 345 from the power source. The electrical switches may be part of the instrument 102 or 202, the control system 106, the auxiliary system 108, or the manipulator assembly 110. The switches may be controlled based on a mode selected by a user. As described above, if a bipolar mode is selected by a user, the switches may be actuated (e.g., under control of the control system 106) to couple the electrode 353 to a power supply line or return line of a bipolar power source and to couple the jaw member 352 to the return line or the power supply line of the bipolar power source. On the other hand, if a monopolar mode is selected, the switches may be actuated to disconnect the electrode 353 from all power sources (including return or ground paths) and to couple the jaw member 352 to a supply line of a monopolar power source.

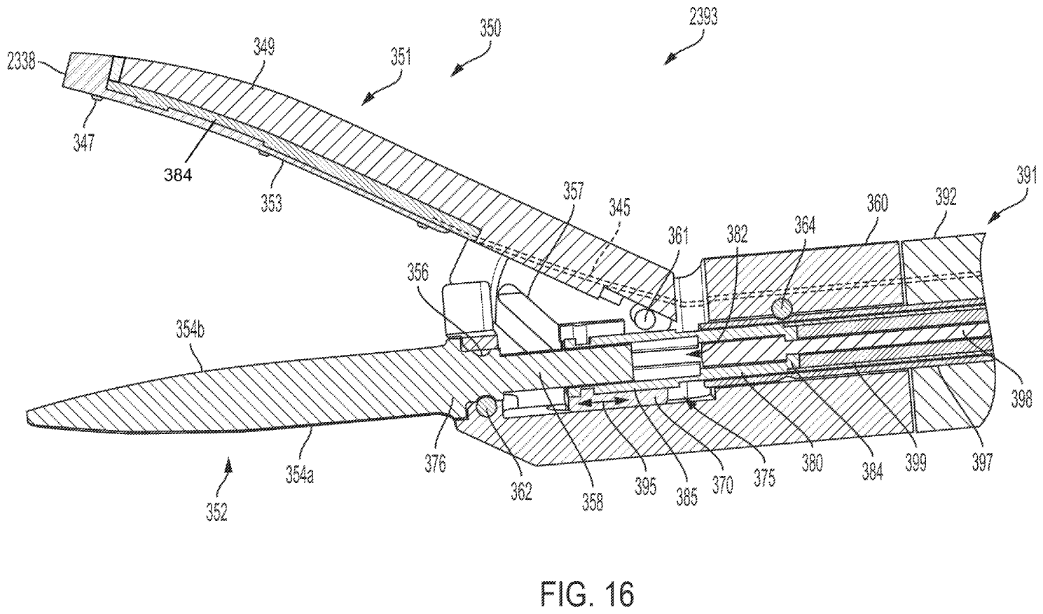

In some embodiments, a monopolar electrode may be provided as part of another portion of the end effector other than the rotatable multifunction jaw member 352. For example, FIG. 16 illustrates an embodiment of an end effector 2393 in which a monopolar electrode 2338 is provided as part of the jaw member 351, which opposes the rotatable multifunction jaw member 352. The end effector 2393 may be similar to the end effector 393 described above, and components of the end effector 2393 are given the same reference numerals in FIG. 16 as identical or similar components of the end effector 393 which were already described above. Duplicative description of these similar components of the end effector 2393 is omitted.

As shown in FIG. 16, the monopolar electrode 2338 is disposed at a distal end of the jaw member 351. The monopolar electrode 2338 may be electrically coupled to the electrode 353. For example, in an embodiment, the monopolar electrode 2338 and the electrode 353 are parts of the same monolithic body. In other embodiments, the monopolar electrode 2338 may be separate from the electrode 353. In FIG. 16, the monopolar electrode 2338 is shown as having a generally rectilinear shape with a relatively blunt or flat end face, but this is non-limiting and other monopolar electrode shapes are contemplated herein. In particular, any shape of electrode may be used for the monopolar electrode 2338. When the monopolar electrode 2338 is being used, the opposing multifunction jaw member 352 may be disconnected from a power source such that the electricity discharged from the monopolar electrode 2338 does not use the multifunction jaw member 352 as a return path. The usage of the monopolar electrode 2338 may be similar to that of the monopolar electrode 1338 described above, except that it is the jaw member 351, rather than the jaw member 352, that is positioned relative to the target tissue. In the illustrated embodiment, the electrode 2338 and the electrode 353 are electrically coupled and therefore electrical power can be supplied to the monopolar electrode 2338 using the electrical power line 345. In other embodiments, the electrode 2338 and the electrode 353 are electrically isolated (e.g., the insulation 348 may extend between the electrode 2338 and the electrode 353), and the electrode 2338 may be coupled to a separate power supply line (not illustrated) that is routed through the jaw member 351 and the clevis 360 in a similar manner as the power line 345. Electrically coupling the electrode 2338 and the electrode 353 together may allow for reduced complexity and reduced size of the end effector 2393, as the second power line and the insulation between the two electrodes can be omitted. On the other hand, electrically isolating the electrode 2338 and the electrode 353 may reduce the possibility of inadvertent discharge from the electrode 2338 into adjacent non-targeted tissue when the electrode 353 is being used in tight spaces.

As with the electrodes 345a and 345b, the electrode 353 may also be configurable between monopolar and bipolar configurations by switching the electrical connections between the various electrodes. By coupling the electrode 353 (via the electrical power line 345) to a power supply path of a monopolar power source while the jaw member 352 is disconnected from the power source, the electrode 353 is configured to operate as a monopolar electrode. By coupling the electrode 353 (via the electrical power line 345) to a power supply path or a return path of a bipolar power source while the jaw member 352 is connected to a return path or power supply path of the bipolar power source, the electrode 353 is configured to operate as a bipolar electrode. Electrical switches, as described above with respect to the embodiment of FIG. 15, may be used to switch the electrical connections to the electrodes 353 and 2338.

The embodiments described herein (including the system 100, instrument 202, end effector 393, multifunction jaw members 452, 552, and 652, and/or force transmission system 1200 described above) may be well suited for use in medical applications. In particular, some embodiments are suitable for use in, for example, surgical, teleoperated surgical, diagnostic, therapeutic, and/or biopsy procedures. Such procedures could be performed, for example, on human patients, animal patients, human cadavers, animal cadavers, and portions or human or animal anatomy. Some embodiments may also be suitable for use in, for example, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein may be used in, or may be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

As used herein, a “functional feature” is a part of a member of an end effector (e.g., a jaw member) that is adapted to a particular function. A functional feature is adapted to a particular function if the functional feature has a configuration (e.g., shape and/or other functional features or attributes) that enables the end effector to perform the particular function or improves the performance of the particular function (e.g., making it more effective, efficient, rapid, or the like). For example, a functional feature for a bipolar sealing function may comprise an electrode (a piece of conductive material coupled to a power source) shaped to have a relatively broad (in a lateral direction) contact surface, while a functional feature for a bipolar cutting function may comprise an electrode having a relatively narrow contact surface. As another example, a functional feature for mechanical cutting may comprise a sharp edge. As another example, a functional feature for securely grasping objects may comprise a contact surface with friction enhancing features (e.g., grooves, protrusions, roughening elements, etc.).

As used herein and in the claims, the term computer-assisted manipulator system (“manipulator system”) should be understood to refer broadly to any system comprising one or more controllable kinematic structures (“manipulators”) comprising one or more links coupled together by one or more joints that can be operated to cause the kinematic structure to move. Such systems may occasionally be referred to in the art and in common usage as robotically assisted systems or robotic systems. The manipulators may have an instrument permanently or removably mounted thereto and may move and operate the instrument. The joints may be driven by drive elements, which may utilize any convenient form of motive power, such as but not limited to electric motors, hydraulic actuators, servomotors, etc. The operation of the manipulator may be controlled by a user (for example through teleoperation), by a computer automatically (so-called autonomous control), or by some combination of these. In examples in which a user controls at least some of the operations of the manipulator, an electronic controller (e.g., a computer) may facilitate or assist in the operation. For example, the electronic controller may “assist” a user-controlled operation by converting control inputs received from the user into electrical signals that actuate drive elements to operate the manipulators, providing feedback to the user, enforcing safety limits, and so on. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems may be implemented in a variety of contexts to perform a variety of procedures, both medical and non-medical. Thus, although some examples described in greater detail herein may be focused on a medical context, the devices and principles described herein are also applicable to other contexts, such as industrial manipulator systems.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes may be made without departing from the spirit and scope of this description and the claims. In some instances, well-known circuits, structures, and techniques have not been shown or described in detail in order not to obscure the embodiments. Like numbers in two or more figures represent the same or similar elements.

Further, the terminology used herein to describe aspects of the invention, such as spatial and relational terms, is chosen to aid the reader in understanding example embodiments of the invention but is not intended to limit the invention. For example, spatially terms—such as “beneath”, “below”, “lower”, “above”, “upper”, “proximal”, “distal”, “up”, “down”, and the like—may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

As used herein, “proximal” and “distal” are spatial/directional terms that describe locations or directions based on their relationship to the two ends of a kinematic chain. “Proximal” is associated with the end of the kinematic chain that is closer to the base or support of the chain, while “distal” is associated with the opposite end of the kinematic chain, which often comprises an end effector of an instrument. Thus, a “proximal” location is a location that is relatively closer to the base of the kinematic chain. For example, the “proximal end portion” of a link refers to the portion of the link that is closer to the base of the kinematic chain than the rest of the link. Conversely, a “distal” location is a location that is relatively farther from the base of the kinematic chain. For example, the “distal end portion” of a link refers to the portion of the link that is farther from the base of the kinematic chain than the rest of the link. The terms closer and farther as used above refer to proximity along the kinematic chain, rather than absolute distance. “Proximal” and “distal” directions are directions that point generally towards a proximal location or distal location, respectively. For example, each link could be described as having associated proximal and distal directions, with the proximal direction of a link pointing generally from around its distal end to around its proximal end and with the distal direction pointing generally from around its proximal end to somewhere around its distal end. It should be understood that for a given kinematic chain there may be many different directions that could be described as “proximal” or “distal” depending on the context, as there may be many links and many possible poses for those links. For example, a “distal” direction described in relation to one link may point diagonally downward relative to a world reference frame, while a “distal” direction described in relation to another link that is at an angle to the first link may point diagonally upward relative to the world reference frame. Moreover, if the poses of the links change, the proximal and distal directions associated with the links may change. Thus, there is no single “proximal” or “distal” direction, but rather many possible “proximal” or “distal” directions, depending on the context. In the context of an instrument attached to the manipulator, “proximal” refers to the end of the instrument attached to the manipulator, while “distal” refers to the opposite end of the instrument which has an end effector. In the context of a surgical procedure, the “distal” end of the kinematic chain is the end that is inserted into a patient, and thus “distal” may also be used to refer to a location that is closer to a patient or to a direction of insertion into a patient, whereas a “proximal” may refer to a location that is further from to the patient or a direction of removal from the patient. In the context of an end effector, the term “proximal” refers to the end of the end effector that is attached to a shaft of the instrument, while “distal” refers to an opposite end of end effector, such as a free end or tip of the end effector. In the context of an end effector, a “proximal-distal axis” refers to an axis that runs between proximal and distal portions of the end effector and that is approximately aligned with a longitudinal axis of the shaft local to the end effector.

As used herein, “transverse” refers to a positional relationship of two items in which one item is oriented crosswise at an angle relative to the other item, such as being substantially or generally perpendicular to the other item. As used herein, “transverse” includes, but does not require, an exactly perpendicular relationship. For example, unless otherwise noted herein or implied by the context, “transverse” may include at least positional relationships in which one item is oriented at an angle between 45° and 135° relative to the other item.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least any inconsequential variations from the value, property, or relationship, such as variations that are equivalents to the stated value, property, or relationship. The range of variation around the stated value, property, or relationship also includes at least those variations that are typical in the relevant art for the type of item in question due to manufacturing or other tolerances. Furthermore, the range of variation also includes at least variations that are within ±5% of the stated value, property, or relationship. Thus, for example, a line or surface may be considered as being “approximately parallel” to a reference line or surface if any one of the following is true: the smallest angle between the line/surface and the reference is less than or equal to 4.5° (i.e., 5% of 90°), the angle is less than or equal to manufacturing or other tolerances typical in the art, or the line/surface as constituted is functionally equivalent to the line/surface if it had been perfectly parallel.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. An instrument comprising:
a shaft; and
an end effector coupled to the shaft, the end effector comprising a jaw mechanism and a rotational coupling, wherein:
the jaw mechanism comprises a first jaw member and a second jaw member movable relative to one another by pivoting about a pivot axis between an open configuration of the jaw mechanism and a closed configuration of the jaw mechanism,
the rotational coupling is operably coupled to the second jaw member and rotatable and translatable relative to the shaft,
translation of the rotational coupling drives movement of the first and second jaw member relative to one another between the closed and open configurations,
rotation of the rotational coupling drives rotation of the second jaw member about a roll axis of the second jaw member between at least two orientations, the roll axis of the second jaw member being transverse to the pivot axis,
in a first orientation of the at least two orientations, the jaw mechanism is configured to perform a first function, and
in a second orientation of the at least two orientations, the jaw mechanism is configured to perform a second function differing from the first function.

2. (canceled)

3. The instrument of claim 1, wherein the first function of the jaw mechanism comprises a bipolar electrical sealing function and the second function of the jaw mechanism comprises a bipolar electrical cutting function.

4. The instrument of claim 3, wherein:
the first jaw member comprises a first electrode portion, and
the second jaw member comprises:
a second electrode portion configured to generate an electrical potential difference with the first electrode portion to provide the bipolar electrical sealing function, and
a third electrode portion configured to generate an electrical potential difference with the first electrode portion to provide the bipolar electrical cutting function.

5. The instrument of claim 1, wherein:
the first jaw member comprises a first electrode portion,
the second jaw member comprises
a second electrode portion having a first contact surface configured to contact material grasped by the jaw mechanism in the first orientation of the second jaw member,
a third electrode portion having a second contact surface configured to contact material grasped by the jaw mechanism in the second orientation of the second jaw member, and
the second contact surface has a larger area than the first contact surface.

6. The instrument of claim 5, wherein:
the second electrode portion and the third electrode portion are integral to a common body.

7. The instrument of claim 5, wherein:
the second electrode portion and the third electrode portion are physically distinct and separated from one another.

8. The instrument of claim 1, wherein:
the first jaw member comprises a first electrode portion,
the second jaw member comprises a second electrode portion positioned opposite from and facing the first electrode portion in the first orientation of the second jaw member, and
the second jaw member comprises a third electrode portion positioned opposite from and facing the first electrode portion in the second orientation of the second jaw member.

9. The instrument of claim 8, wherein:
the second electrode portion and the third electrode portion are integral to a common body.

10. The instrument of claim 9, wherein:
the second electrode portion and the third electrode portion are physically distinct and separated from one another.

11. The instrument of claim 8, wherein:
the first and second electrode portions are configured to generate an electrical potential difference to perform an electrosurgical sealing function in the first orientation of the second jaw member, the electrosurgical sealing function being the first function, and
the first and third electrode portions are configured to generate an electrical potential difference to perform an electrosurgical cutting function in the second orientation of the second jaw member, the electrosurgical cutting function being the second function.

12. The instrument of claim 1, wherein:
the rotational coupling is translatable relative to the second jaw member and operably coupled to the second jaw member such that the second jaw member is constrained to rotate with rotation of the rotational coupling.

13. (canceled)

14. (canceled)

15. The instrument of claim 12, further comprising:
a closure block operably coupled to the first jaw member and to the rotational coupling,
wherein the closure block converts translation of the rotational coupling into pivoting of the first jaw member to move the jaw mechanism between the open and closed configurations.

16. The instrument of claim 15, wherein:
the rotational coupling is rotatable relative to the closure block, and
the closure block is constrained to translate with the rotational coupling.

17. The instrument of claim 12, further comprising:
an actuatable element extending through the shaft and operably coupled to the rotational coupling, the actuatable element configured to transmit force to drive rotation and translation of the rotational coupling relative to the shaft.

18. The instrument of claim 1, wherein:
the first and second functions of the jaw mechanism are chosen from a bipolar electrical sealing function, a bipolar electrical cutting function, a mechanical cutting function, or a grasping function.

19. The instrument of claim 1, wherein:
the at least two orientations of the second jaw member comprise three or more orientations each corresponding to a differing function of the jaw mechanism.

20-26. (canceled)

27. An instrument comprising:
a shaft;
an actuatable element extending through the shaft; and
an instrument transmission system coupled to the shaft and comprising:
a chassis;
a first drive input;
a second drive input;
a first gear assembly operably coupled to and driven by the first drive input; and
a second gear assembly operably coupled to and driven by the second drive input;
wherein rotation of the first gear assembly and the second gear assembly relative to the chassis at a same angular velocity causes rotation of the actuatable element relative to the shaft; and
rotation of the first gear assembly relative to the chassis while the second gear assembly is held stationary relative to the chassis causes translation of the actuatable element relative to the shaft.

28. The instrument of claim 27, wherein:
rotation of the first gear assembly and the second gear assembly relative to the chassis at a same angular velocity causes rotation of the actuatable element relative to the shaft independent of any translation of the actuatable element relative to the shaft, and
rotation of the first gear assembly relative to the chassis while the second gear assembly is stationary relative to the chassis causes translation of the actuatable element relative to the shaft independent of any rotation of the actuatable element relative to the shaft.

29. The instrument of claim 27, further comprising:
an end effector coupled to the shaft,
wherein translation of the actuatable element imparts a first degree of freedom movement to the end effector relative to the shaft, and
wherein rotation of the actuatable element imparts a second degree of freedom movement, different from the first degree of freedom movement, to the end effector relative to the shaft.

30. A method of using an instrument that has a jaw mechanism, comprising:
pivoting a first jaw member and a second jaw member of the jaw mechanism about a pivot axis between closed and open configurations by translating a rotational coupling operably coupled to the second jaw member;
while a first jaw member of the jaw mechanism is in a first orientation, causing the first jaw member to perform a first function on material grasped by the jaw mechanism;
rotating the first jaw member from the first orientation to a second orientation about a rotation axis extending from a proximal end of the first jaw member to a distal end of the first jaw member by rotating the rotational coupling; and
while the first jaw member is in the second orientation, causing the first jaw member to perform a second function, different from the first function, on material grasped by the jaw mechanism.
